(19) United States
(12) Patent Application Publication
(10) Pub. No.: US 2024/0091386 A1
(43) Pub. Date: Mar. 21, 2024

(54) COMPOSITIONS, METHODS, MODELS AND USES FOR SIMIAN VARICELLA VIRUS (SVV) CHIMERIC CONSTRUCTS IN HUMAN HEALTH CONDITIONS

(71) Applicants: THE REGENTS OF THE UNIVERSITY OF COLORADO, A BODY CORPORATE, Denver, CO (US); THE ADMINISTRATORS OF THE TULANE EDUCATIONAL FUND, New Orleans, LA (US); RUTGERS, THE STATE UNIVERSITY OF NEW JERSEY, New Brunswick, NJ (US)

(72) Inventors: Ravi Mahalingam, Aurora, CO (US); Vicki L. Traina-Dorge, Covington, LA (US); Hua Zhu, Plainsboro, NJ (US)

(21) Appl. No.: 17/930,234

(22) Filed: Sep. 7, 2022

Related U.S. Application Data
(63) Continuation of application No. PCT/US2021/023769, filed on Mar. 23, 2021.
(60) Provisional application No. 62/993,400, filed on Mar. 23, 2020.

(51) Int. Cl. A61K 49/00; C07K 14/005; C12N 7/00

(52) U.S. Cl. CPC A61K 49/0008; C07K 14/005; C12N 7/00; C12N 2710/16721; C12N 2710/16722; C12N 2710/16751

(57) ABSTRACT

Embodiments of the instantly claimed inventions include, but are not limited to, chimeric viral constructs, non-human primate models, in vivo screening systems for antiviral agents, gene therapy delivery systems, and methods of making and using the same. In some embodiments, chimeric viral constructs include a non-human primate infecting virus nucleic acid sequence and a exclusively human pathogenic virus nucleic acid sequence for use in creating a non-human primate model and uses thereof. In other embodiments, systems for testing antiviral agents are disclosed. In other embodiments, gene therapy delivery systems disclosed herein can be used to deliver a vector containing or associated with an agent to a human subject for treating conditions of the skin and neuronal ganglia in the subject.

### PRIORITY

This U.C. Continuation Applications claims the benefit of International Application PCT/US2021/023769, filed Mar. 23, 2021 which application claims priority to U.S. Provisional Application No. 62/993,400, filed Mar. 23, 2020. These applications are incorporated herein by reference in their entirety for all purposes.

### STATEMENT REGARDING SEQUENCE LISTING

The instant application contains a Sequence Listing which has been submitted via ASCII copy created on Mar. 23, 2021 referred to as ‘106549-739332 CU4813H-US1 Sequence Listing.xml’ of size 894 Kilobytes having 18 sequences.

### FIELD

Embodiments of disclosed herein concern novel chimeric viral constructs, non-human primate models, in vivo screening systems for antiviral drugs and vaccines, gene therapy delivery systems, and methods of making and use thereof. In some embodiments, methods of making chimeric viral constructs of use as a platform construct are disclosed. In other embodiments, non-human primate models are described having chimeric viral constructs disclosed herein of use for in vivo testing human vaccines and antiviral agents. In other embodiments, gene therapy delivery systems are disclosed. In some embodiments, gene therapy delivery systems disclosed herein can be used to treat a disease, an infection and/or a condition in a subject in need thereof. In some embodiments

[symbol]

### BACKGROUND

Varicella zoster virus (VZV) is a member of the herpesvirus family that causes chickenpox in non-immune hosts. Primary VZV infection can be followed by latency in ganglionic neurons. During the latency period, little to no VZV particles are produced and no obvious neuronal damage occurs. Reactivation of VZV leads to virus replication which can cause zoster (shingles) in tissues innervated by the involved neurons, inflammation and cell death—a process that can lead to persistent radicular pain (e.g., postherpetic neuralgia (PHN)). Zoster-induced pain can occur without evidence of skin rash and older patients often develop PHN. Additionally, reactivation of VZV can elevate risk for other side effects such as stroke, dementia, Alzheimer's disease, and cognitive impairment due to reactivated VZV.

There are a limited number of vaccines available for VZV; however, immunity to varicella does not protect against reactivation of the virus. One of the barriers to developing antivirals that treat reactivated herpesviruses, such as VZV, and/or vaccines that protect against VZV infection of the nervous system is that an animal model for analyzing efficacy of vaccines in humans does not exist.

### SUMMARY

Embodiments disclosed herein concern novel chimeric viral constructs. In some embodiments, chimeric viral constructs as disclosed herein include a nucleic acid sequence or polynucleotide derived from an exclusively human pathogenic virus, and a nucleic acid sequence fragment or polynucleotide of simian varicella virus (SVV). In some embodiments, the nucleic acid sequence fragment of SVV of use in compositions and methods disclosed herein can have about 85% or more sequence identity to SEQ ID NO: 1.

In some embodiments, a chimeric viral construct disclosed herein can include a nucleic acid sequence from an exclusively human pathogenic virus. In other embodiments, a nucleic acid sequence from an exclusively human pathogenic virus can be a nucleic acid sequence of an adenovirus, an anellovirus, an astrovirus, a calicivirus, a coronavirus, a flavivirus, a herpesvirus, an orthomyxovirus, a papillomavirus, a paramyxovirus, a parvovirus, a picornavirus, a polyomavirus, a poxvirus, a pneumovirus, a retrovirus, or any combination thereof. In other embodiments, a nucleic acid sequence from an exclusively human pathogenic virus can be a nucleic acid sequence from a herpesvirus. In some embodiments, the polynucleotide or nucleic acid sequence can include the full length sequence or an infective fragment thereof. In certain embodiments, a nucleic acid sequence from a herpesvirus can include a nucleic acid sequence of human herpes simplex virus type 1 (HSV-1), herpes simplex virus type 2 (HSV-2), Epstein-Barr-virus (EBV), cytomegalovirus (CMV), human herpes virus type 6 (HHV-6), human herpes virus type 7 (HHV-7), human herpes virus type-8 (HHV-8), varicella zoster virus (VZV), or any combination thereof. In certain embodiments, a nucleic acid sequence from an exclusively human pathogenic virus can be a nucleic acid sequence derived from VZV.

Some embodiments disclosed herein concern methods of making any one of the chimeric viral constructs disclosed herein. In some embodiments, methods of making a chimeric viral constructs disclosed herein can include using a recombineering system to introduce the nucleic acid sequence fragment of SVV into one or more locations within the nucleic acid sequence from an exclusively human pathogenic virus. In some embodiments, chimeric viral constructs herein can include, but are not limited to, obtaining a nucleic acid sequence fragment of SVV and introducing this fragment into at least one of an N-terminus of the nucleic acid sequence from an exclusively human pathogenic virus, a C-terminus of the nucleic acid sequence from an exclusively human pathogenic virus, one or more locations between the N-terminus and the C-terminus of the nucleic acid sequence from an exclusively human pathogenic virus, or any combination. In accordance with these embodiments, the insertion of the SVV nucleic acid fragment into the exclusively human pathogenic virus does not disrupt expression of the human pathogenic virus in the non-human primate model permitting infectivity of the non-human pathogenic virus. In some embodiments, methods of producing a chimeric viral constructs disclosed herein can include obtaining a nucleic acid sequence fragment of SVV and inserting it into at least one of an N-terminus of the nucleic acid sequence of a human VZV, a C-terminus of the nucleic acid sequence of a human VZV, a unique short sequence (U_{S}) of the nucleic acid sequence of a human VZV, a nucleic acid segment between open reading frame 65 (ORF65) and open reading frame 66 (ORF66) of the nucleic acid sequence of a human VZV, or any combination thereof. In accordance with these embodiments, the insertion of the SVV nucleic acid fragment into the human VZV virus does not disrupt expression of the human VZV virus in the non-human primate model permitting infectivity of the non-human pathogenic virus.

Some embodiments disclosed herein concern non-human primate models, methods of creating the model and methods of use for studying exclusively human pathogenic viral disease side effect (e.g. acute or chronic side effect) or condition. In some embodiments, a non-human primate model herein can be a non-human primate infected with one or more chimeric viral constructs disclosed herein adapted for expression and infection in the non-human primate. In some embodiments, a non-human primate model of use herein can be a rhesus macaque, a cynomolgus macaque, an African green monkey, a baboon, a sooty mangabey, a common marmoset, a capuchin, an owl monkey, a patas money, a pigtail macaque, a sabaeus monkey, a squirrel monkey, a tamarin, or any combination thereof. In certain embodiments, the non

[symbol]

In some embodiments, a non-human primate model as disclosed herein can be infected with a chimeric viral construct having a nucleic acid sequence of a human VZV and a nucleic acid sequence fragment of SVV sharing about 85% or more sequence identity to SEQ ID NO: 1. In other embodiments, a non-human primate model as disclosed herein can be infected with a chimeric viral construct having a nucleic acid sequence of a human VZV and a nucleic acid sequence fragment of SVV sharing about 85% or more sequence identity to SEQ ID NO: 1 wherein the nucleic acid sequence of a human VZV can have at least 3 stop codons in open reading frame 7 (ORF7). In yet other embodiments, the VZV region of these exemplary constructs can be replaced with another different exclusively human pathogenic virus nucleic acid in order to create a model for studying the different exclusively human pathogenic virus.

In some embodiments, methods of making non-human primate models can include infecting any one of the non-human primates disclosed herein with one or more of the chimeric viral constructs herein. In some embodiments, methods of making non-human primate models can include infecting any one of the non-human primates disclosed herein with one or more of the chimeric viral constructs herein with an amount sufficient to induce at least one symptom of infection caused by the exclusively human pathogenic virus. In some embodiments, at least one symptom of infection can be a clinical symptom, a pathological symptom, a cognitive symptom, or any combination thereof. In other embodiments, the at least one symptom of the infection parallels a symptom of a human host.

Other embodiments disclosed herein concern in vivo screening systems for antiviral agents such as antiviral drugs and vaccines and methods of use. In certain embodiments, antiviral agents can include general antiviral agents of use against multiple viruses. In other embodiments, vaccines can include viral vaccines of use for a general vaccine against viral infection or a specific vaccine to an exclusively human pathogenic virus. In other embodiments, in vivo screening systems for antiviral agents such as antiviral drugs and vaccines disclosed herein can include: a non-human primate model infected with a chimeric viral construct disclosed herein and at least one antiviral agent such as an antiviral drug or vaccine for use in testing in the in vivo screening system. In some embodiments, in vivo screening systems for antiviral agents disclosed herein can further include reagents for measuring at least one positive and negative output for analyzing and assessing efficacy of the antiviral agent using the in vivo screening system. In some embodiments, chimeric viral constructs herein can be of use as a platform construct according to certain embodiments of the disclosure.

In some embodiments, methods of using in vivo screening systems disclosed herein can include one or more of the following: administering at least one antiviral agent such as an antiviral drug or vaccine to a non-human primate model infected with a chimeric viral construct disclosed herein; monitoring the non-human primate model infected with the chimeric viral construct for one or more at least one symptom of infection by the exclusively human pathogenic virus, wherein at least one symptom of infection can be a clinical symptom, a pathological symptom, a cognitive symptom, or any combination thereof. In some embodiments, methods of using in vivo screening systems disclosed herein can further include collecting at least one biological sample from the non-human primate after administration of the at least one antiviral agent such as an antiviral drug or vaccine and subjecting the biological sample to at least one assay, wherein the assay can measure at least one of positive and negative outputs from the in vivo screening system. In certain embodiments, the in vivo screening systems disclosed herein can be used to assess efficacy of an antiviral agent (e.g. vaccine or general antiviral agent) against early or latent exclusively human pathogenic viruses to measure reduction in symptoms, prevention or attenuation of infection or other relevant parameter for assessing efficacy of the agent.

Some embodiments disclosed herein concern gene therapy delivery systems and methods of use. In some embodiments, gene therapy delivery systems can include: a nucleic acid sequence of open reading frame 7 (ORF7) of human VZV; a nucleic acid sequence encoding at least one therapeutic gene, wherein the at least one therapeutic gene can be expressed in a neuronal cell, a dermal cell, or any combination thereof. In some embodiments, gene therapy delivery systems herein can include a nucleic acid sequence of open reading frame 7 (ORF7) of human VZV wherein ORF7 can have about 85% or more sequence identity to SEQ ID NO: 8. In some embodiments, gene therapy delivery systems herein can include a nucleic acid sequence of open reading frame 7 (ORF7) of human VZV wherein ORF7 encodes for a protein that can have about 85% or more sequence identity to SEQ ID NO: 9. In some embodiments, gene therapy delivery systems herein can include a viral vector. In some embodiments, gene therapy delivery systems herein can include a viral vector, wherein the viral vector can be a retrovirus, an adenovirus, an adeno-associated virus, a lentivirus, a pox virus, an alphavirus, a herpes virus, or any combination thereof. In some embodiments, gene therapy delivery systems herein can include a non-viral vector. In some embodiments, gene therapy delivery systems herein can include a non-viral vector, wherein the non-viral vector can be naked DNA, an oligonucleotide, a lipoplex, a polyplex, or any combination thereof. In some embodiments, gene therapy delivery systems herein can further include at least one pharmaceutically acceptable carrier.

In some embodiments, gene therapy delivery systems disclosed herein can be used in methods of treating a disease or a condition in a subject. In accordance with some embodiments, any one of the wherein gene therapy delivery systems herein can be administered to a subject having a disease or a condition in need of treatment. In some embodiments, gene therapy delivery systems disclosed herein can be administered to a subject by subcutaneous injection, intravenous injection, intracerebroventricular injection, intra-cisterna magna injection, intra-parenchymal injection, or a combination thereof.

In certain embodiments, the present disclosure provides for kits including, but not limited to, one or more chimeric viral constructs as disclosed herein and at least one container. In some embodiments, kits disclosed herein can further include a device for delivering the one or more chimeric viral constructs to at least one non-human primate. In some embodiments, kits herein can be used to practice any of the methods disclosed herein including, but not limited to, screening for at least one antiviral drug, at least one vaccine, or any combination thereof, for the treatment of an exclusively human pathogenic virus; screening for at least one symptom of infection by an exclusively human pathogenic virus, wherein the at least one symptom of infection can include a clinical symptom, a pathological symptom, a cognitive symptom, or any combination thereof; and administering at least one therapeutic gene to be expressed in a neuronal cell, a dermal cell, or any combination thereof.

### BRIEF DESCRIPTION OF THE FIGURES

The following drawings form part of the present specification and are included to further demonstrate certain embodiments of the present disclosure. Certain embodiments can be better understood by reference to one or more of these drawings in combination with the detailed description of specific embodiments presented herein.

FIG. 1 illustrates a comparison of left end VZV and SVV gene segments in accordance with certain embodiments of the present disclosure.

FIGS. 2A and 2B illustrate examples of chimeric constructs of VZV and SVV v in accordance with certain embodiments of the present disclosure.

FIGS. 3A to 3D illustrate examples of chimeric constructs referenced as SVZV chimeric viruses in accordance with certain embodiments of the present disclosure.

FIGS. 4A to 4D illustrate examples of chimeric constructs referenced as SVZV chimeric viruses (4A), and examples illustrating plaques formed when the constructs were transfected into a cell line (4B). 4C illustrates an example agarose gel demonstrating presence of fragments of constructs, and 4D illustrates a graphical representation of an exemplary growth curve analysis of SVZV chimeric viruses in a cell line (4D) in accordance with certain embodiments of the present disclosure.

FIGS. 5A and 5B illustrate photograph of a removed mouse kidney (5A) of an exemplary experiment in comparison to a kidney (5B) from a mouse model having human dorsal root ganglion (DRG) implanted in accordance with certain embodiments of the present disclosure.

FIGS. 5C and 5D illustrate an example radiograph (5A) and line plot (5B) illustrating viral growth over a certain period in a mouse model having been infected with exemplary chimeric viruses (VZV with an intact ORF7 (WT and R7) and VZV with an ORF7 having stop codons (D7)) in accordance with certain embodiments of the present disclosure.

FIGS. 5E and 5F illustrate example immunohistochemistry images (5E) and fluorescence in-situ hybridization (FISH) images (5F) of implanted ganglia from a mouse model having been infected with exemplary chimeric viruses (VZV with an intact ORF7 (WT) and VZV with an ORF7 having stop codons (D7)) in accordance with certain embodiments of the present disclosure.

FIGS. 5G and 5H illustrate an example of a merged image (fluorescent over phase-contrast) of ex vivo DRG culture (5G) or in vitro of a cancer cell line (5H) infected with exemplary chimeric viruses (VZV with an intact ORF7 (rOka) and VZV with an ORF7 having stop codons (rOka-D7)) in accordance with certain embodiments of the present disclosure.

FIGS. 6A to 6K illustrate examples of a fluorescent image (6A-6B) and an electron micrograph image at different magnifications (6C to 6K, insets in 6C, 6F and 6I are enlarged in 6D, 6E, 6G, 6H, 6J and 6K as indicated) of cells infected with an exemplary chimeric virus (VZV with an intact ORF7 (rOka) and VZV with an ORF7 having stop codons (rOka-D7)) in vitro, (6A and 6C-6K) and in a microfluidic chamber (6B) in accordance with certain embodiments of the present disclosure.

FIG. 7 illustrates exemplary growth curves of exemplary SVZV chimeric virus in comparison to a control wild type VZV virus in representative cell types in accordance with certain embodiments of the present disclosure.

FIGS. 8A and 8B illustrate exemplary growth curves (8A) and exemplary fluorescent images (8B) of demonstrating two experimental SVZV chimeras versus control (SVZV-N) VZV virus (VZV-WT) and SVZV with ORF7 with stop codons (SVZV-N-7D) growth post infection as plotted and demonstrated in culture in accordance with certain embodiments of the present disclosure.

FIGS. 9A and 9B illustrate exemplary growth curves (9A) and exemplary fluorescent images (9B) of cultured cells post-infection of SVZV-N chimeras and SVZV with ORF7 with stop codons (SVZV-N-7D) or VZV control viruses (VZV-WT) in accordance with certain embodiments of the present disclosure.

FIGS. 10A and 10B illustrate example immunohistochemistry images of SVZV-NN infected African Green monkey skin tissue stained with either rabbit anti-VZV (10A) or normal rabbit serum (10B) in accordance with certain embodiments of the present disclosure.

### DETAILED DESCRIPTION

In the following sections, various exemplary compositions and methods are described in order to detail various embodiments of the disclosure. It will be obvious to one of skill in the relevant art that practicing the various embodiments does not require the employment of all or even some of the details outlined herein, but rather that concentrations, times and other details can be modified through routine experimentation. In some cases, well-known methods or components have not been included in the description.

Embodiments of the instant disclosure relate to compositions, methods, and kits including, but not limited to, chimeric viral constructs, non-human primate models, in vivo screening systems for antiviral agents such as antiviral drugs and vaccines for general or specific prevention or treatment of viral infections, and gene therapy delivery systems. Other embodiments of the instant disclosure relate to methods of making chimeric viral constructs, non-human primate models, in vivo screening systems for testing antiviral agents against exclusively human pathogenic viruses such as antiviral drugs and vaccine against an exclusively human pathogenic virus. Other embodiments of the instant disclosure relate to methods of directing an agent or gene to specified tissues or cells of a human subject. In accordance with these embodiments, compositions and methods are disclosed for gene therapy treatments or delivering an agent of interest to the specified tissue using time-release or other technology. In yet other embodiments, methods for creating and using gene therapy delivery systems are disclosed herein of use to treat a disease a disease or a condition in a subject in need thereof.

Embodiments of the instant disclosure relate to compositions and methods for creating chimeric viral constructs. As used herein, the term “chimeric viral constructs” refers to a nucleic acid construct encoding for a virus having a genome containing sequences from two or more different viruses, including different viral strains where the two or more different viruses are linked or associated in a manner for concurrent expression, for example.

In some embodiments, chimeric viral constructs disclosed herein include a nucleic acid sequence from an exclusively human pathogenic virus in combination with another virus. As used herein, the term “exclusively human pathogenic virus” refers to a virus known to naturally infect only human subjects to cause disease. In some embodiments, chimeric viral constructs herein can have a nucleic acid sequence from an exclusively human pathogenic virus where the virus can be an adenovirus, an anellovirius, an astrovirus, a calicivirius, a coronavirus, a flavivirus, a herpesvirus, an orthomyxovirus, a papillomavirus, a paramyxovirus, a parvovirus, a picornavirus, a polyomavirus, a poxvirus, a pneumovirus, a retrovirus, or any combination thereof. Non-limiting examples of viruses exclusively infecting and causing disease in a human host can include Aichi virus, BK polyomavirus, Cosavirus A, Coxsackievirus, Echovirus, Epstein-Barr virus, GB virus C/Hepatitis G virus, Hepatitis A virus, Hepatitis C virus, Hepatitis delta virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human spumaretrovirus, Human T-lymphotropic virus, Human torovirus, Influenza B virus, Influenza C virus, JC polyomavirus, KI Polyomavirus, Lordsdale virus, Measles virus, Merkel cell polyomavirus, Molluscum contagiosum virus, Mumps virus, Norwalk virus, Rosavirus A, Rotavirus A, Rotavirus B, Rotavirus C, Rubella virus, Salivirus A, Sapporo virus, Southampton virus, Torque teno virus, Varicella-zoster virus, Variola virus, or any strain variant thereof.

In some embodiments, chimeric viral constructs herein can have a nucleic acid sequence from a herpesvirus. In some embodiments, chimeric viral constructs herein having a nucleic acid sequence from a herpesvirus can have a nucleic acid sequence of human herpes simplex virus type 1 (HSV-1), herpes simplex virus type 2 (HSV-2), Epstein-Barr-virus (EBV), cytomegalovirus (CMV), human herpes virus type 6 (HHV-6), human herpes virus type 7 (HHV-7), human herpes virus type-8 (HHV-8), varicella zoster virus (VZV), or any combination thereof. In some embodiments, chimeric viral constructs herein can have nucleic acid sequence from a herpesvirus can have a nucleic acid sequence of VZV. In some embodiments, chimeric viral constructs herein can have a full-length, truncated, or fragment of a nucleic acid sequence of VZV. One of skill in the can appreciate that a because a virus can have multiple strains within one virus type, a nucleic acid sequence as used herein can share 85% or more (e.g., about 85%, about 90%, about 95%, about 99%, about 100%) sequence identity among the strains within the one virus type. In accordance with these embodiments, chimeric viral constructs herein can include, but are not limited to, a nucleic acid sequence of VZV sharing 85% or more (e.g., about 85%, about 90%, about 95%, about 99%, about 100%) sequence identity with the VZV genome (e.g. NCBI Reference Sequence No. NC_001348.1 or equivalent nucleic acid sequence thereof). In accordance with these embodiments, chimeric viral constructs herein can include, but are not limited to, a nucleic acid sequence of VZV sharing 85% or more (e.g., about 85%, about 90%, about 95%, about 99%, about 100%) sequence identity with the VZV genome, SEQ ID NO: 17. In accordance with these embodiments, it is contemplated that the exclusively human pathogenic viruses of use in constructs disclosed herein have more than one representative sequence where sequences can vary and all of these various forms of the exclusively human pathogenic viruses are contemplated of use herein.

In some embodiments, chimeric viral constructs herein can have a nucleic acid sequence from an exclusively human pathogenic virus wherein one or more nucleotides of the sequence have been modified while preserving its infectivity. In accordance with some embodiments, insertions and deletions are contemplated. In other embodiments, modification of one or more nucleotides can include, but is not limited to: deletion of one or more nucleotides from the nucleic acid sequence; inserting one or more nucleotides into the nucleic acid sequence; changing one or more nucleotides of a naturally occurring nucleic acid sequence to one or more nucleotides of a non-naturally occurring nucleic acid sequence (e.g., A→T; A→G; A→C; T→A; T→G; T→C; C→A; C→G; C→T; G→A; G→T; G→C). In accordance with some embodiments, modification of nucleic acid sequences herein can be performed using one or more techniques known in the art (e.g., site-directed mutagenesis, seamless ligation cloning extract (SLiCE), homologous recombination, CRISPR gene editing).

In some embodiments, chimeric viral constructs herein can include a nucleic acid sequence of a herpesvirus where one or more nucleotides have been modified while conserving infectivity capabilities in a host. In accordance with these embodiments, one or more modifications can include a conservative substitution, an insertion or deletion. In some embodiments, chimeric viral constructs disclosed herein can include a nucleic acid sequence of VZV wherein one or more nucleotides have been modified. In accordance with these embodiments, a nucleic acid sequence of VZV as used herein can be modified at one or more sites in the VZV genome while preserving VZV infectivity in a host such as a human host or a non-human primate model disclosed herein. In some embodiments, a nucleic acid sequence of VZV of use herein can be modified at one or more ORFs in the VZV genome. In some embodiments, a nucleic acid sequence of VZV as used herein can be modified at ORF7 in the VZV genome. In some embodiments, a nucleic acid sequence of VZV as used herein can be modified at a nucleic acid sequence of VZV ORF7 sharing 85% or more (e.g., about 85%, about 90%, about 95%, about 99%, about 100%) sequence identity to SEQ ID NO: 8. In some embodiments, a nucleic acid sequence of VZV as used herein can be modified at a nucleic acid sequence of VZV ORF7 such that the nucleic acid sequence encodes for a protein sharing 85% or more (e.g., about 85%, about 90%, about 95%, about 99%, about 100%) sequence identity to SEQ ID NO: 9.

In some embodiments, a nucleic acid sequence of VZV of use herein can be modified to introduce one or more stop codons (e.g., about one, about two, about three, about four stop codons) into the ORF7 of the VZV genome. In some embodiments, a nucleic acid sequence of VZV herein can be modified to introduce stop codons into the ORF7 of the VZV genome sharing 85% or more (e.g., about 85%, about 90%, about 95%, about 99%, about 100%) sequence identity to 5′-TAGC TGACTAAGTGTGCCAGCTTATGTGGATATG-3′ (SEQ ID NO: 10). In some embodiments, a nucleic acid sequence of VZV herein modified to introduce one or more stop codons into the ORF7 of the VZV genome can have a ORF7 nucleic acid sequence sharing 85% or more (e.g., about 85%, about 90%, about 95%, about 99%, about 100%) sequence identity to SEQ ID NO: 8, as provided below:

(SEQ ID NO: 8)
ATGC AGACGGTGTG TGCCAGCTTA TGTGGATATG
CTCGAATACC AACTGAAGAG CCATCTTATG AAGAGGTGCG
TGTAAACACG CACCCCCAAG GAGCCGCCCT GCTCCGCCTC
CAAGAGGCTT TAACCGCTGT GAATGGATTA TTGCCTGCAC
CTCTAACGTT AGAAGACGTA GTCGCTTCTG CAGATAATAC
CCGTCGTTTG GTCCGCGCCC AGGCTTTGGC GCGAACTTAC
GCTGCATGTT CTCGTAACAT TGAATGTTTA AAACAGCACC
ATTTTACTGA AGATAACCCC GGTCTTAACG CCGTGGTCCG
TTCACACATG GAAAACTCAA AACGGCTTGC TGATATGTGT
TTAGCTGCAA TTACCCATTT GTATTTATCG GTTGGCGCGG
TGGATGTTAC TACGGATGAT ATTGTCGATC AAACCCTGAG
AATGACCGCT GAAAGTGAAG TGGTCATGTC TGATGTTGTT
CTTTTGGAGA AAACTCTTGG GGTCGTTGCT AAACCTCAGG
CATCGTTTGA TGTTTCCCAC AACCATGAAT TATCTATAGC
TAAAGGGGAA AATGTGGGTT TAAAAACATC ACCTATTAAA
TCGGAGGCGA CACAATTATC TGAAATTAAA CCCCCACTTA
TAGAAGTATC GGATAATAAC ACATCTAACC TAACAAAAAA
AACGTATCCG ACAGAAACTC TTCAGCCCGT GTTGACCCCA
AAACAGACGC AAGATGTACA ACGCACAACC CCCGCGATCA
AGAAATCCCA TGTTATGCTT GTATAAATAT TGAAATAAA.

In some embodiments, a nucleic acid sequence of VZV herein can be modified to introduce one or more stop codons into the ORF7 of the VZV genome, wherein the modified nucleic acid sequences can encode for one or more of the following polypeptides:

5′-Stop L T K C A S L C G Y-3′
5′-S Stop L S V P A Y V D M-3′
5′-A D Stop V C Q L Met W I-3′

In some embodiments, chimeric viral constructs herein can have a nucleic acid sequence from a simian varicella virus (SVV). In accordance with some embodiments, chimeric viral constructs herein can have a nucleic acid sequence of SVV sharing 85% or more (e.g., about 85%, about 90%, about 95%, about 99%, about 100%) sequence identity with the SVV genome (NCBI Reference Sequence No. NC_002686.2 or equivalent nucleic acid sequence thereof). In some embodiments, chimeric viral constructs herein can have a nucleic acid sequence from a simian varicella virus (SVV). In accordance with some embodiments, chimeric viral constructs herein can have a nucleic acid sequence of SVV sharing 85% or more (e.g., about 85%, about 90%, about 95%, about 99%, about 100%) sequence identity with the SVV genome, SEQ ID NO: 18.

In some embodiments, chimeric viral constructs herein can have a nucleic acid sequence fragment of SVV. As used herein, a “nucleic acid sequence fragment” can refer to any portion of a genome sequence, wherein the fragment is not the entire genome sequence. In some embodiments, a nucleic acid sequence fragment of SVV herein can be about 0.01 kilobases (kb) to about 3.5 kb (e.g., about 0.01 kb, about 0.1 kb, about 1 kb, about 1.5 kb, about 2.0 kb, about 2.5 kb, about 3.0 kb, about 3.5 kb). In accordance with some embodiments, a nucleic acid sequence fragment of SVV herein can be about 3.0 kb.

In some embodiments, a nucleic acid sequence fragment of SVV herein can have about 1 nucleotide to about 4000 nucleotides (e.g., about 1 nucleotide, about 50 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1000 nucleotides, about 1500 nucleotides, about 2000 nucleotides, about 2500 nucleotides, about 3000 nucleotides, about 3500 nucleotides, about 4000 nucleotides). In accordance with some embodiments, a nucleic acid sequence fragment of SVV herein can be about 3600 nucleotides. In some embodiments, a nucleic acid sequence fragment of SVV herein can have a nucleic acid sequence of SVV (e.g., nucleotides 1-3600) sharing 85% or more (e.g., about 85%, about 90%, about 95%, about 99%, about 100%) sequence identity to SEQ ID NO: 1, provided below:

(SEQ ID NO: 1)
CCAGAACGTCATGTTTTTATTCCGCTTTCATCGTCTGCATT
GTCGTCTCTCCAATCTATCGCTCCTCCCACACCCCCTACT
TACGATCGGACGTGTGCCATTGCCTCCAATCCACCTACTT
ACGAACATGCTCCACCCCCAACTTACGAACAGGCGTGTGC
CTTTGGTTCCAATTCTTCGCTTCCAGATCTTAGGATAGAA
CCGAGGACAGAACCGAGGACGGATCTGCCCCCAGCTTACA
CTTCCGATACACATGCAACGCATAACGTCCCGATGGAAAC
GTTACAGTGTTGTAACTCTGCTCATTCTGCTGTGTGGTTA
CAGGATGTCATAGTTGCACGTATTCGGAGATTTATACCGC
CTTGTCTTGTTTTGGGACTTGTGGTCGTTGCAGCCATTAT
ATTACTTCTAATTTCACTCAATCCCCATTAACTTTCAGTT
TTAATAAAACATGCGGGTATTTTTTTACACTGTTTATCGC
TGCGTTTTACTTTTTTTTTTTCTATTTCTCCACACATGTT
ACTCTACCCGCGCTATACTCCCTTATCTAAACCCTTTGTA
CCCGCGGTTCGTACGCATAAACACAATGTTGCTACGTTTC
ACTGTCATCCATTTTAACCTTTATTTTCCTTTTACATTAC
AGACACGTTCGCTTAGAAGTTTTTACGTTTTATTATTTGC
AATATATGTTAAACTCGGAACACCGAAAGAATTTACCAAA
CTTTTCCGTGTGATGCACATACTCTGGCCAGCATACAATT
TTACACTCTACTTTATTGCAATATCTTCCGTGATGCATTA
GCGCCCCCAACACGCCGGCCTCACATGCCCCGGGTGTATA
GTCTTCGAGCACATTACAAGACTCGCTTACGTTCTCATAT
GCAATATGTTCATCGCCTCGTTGCACCGCGGTTACAATTC
GTGCAACTGTAACAAACATTAACGTTGTGATACACGTAAT
GTCATTAAAACTTTGCCGCTCTAGTAACTCTACCAACGTC
GTGGTCTCCGCCCTTTGTTGTGTCAATATGCATTTCACCA
TGGGTTCCAATTTTAACTTCAAGTTGTCTAACAACACGCT
TGAGGTCGCAACAATGGGGTTCCACGTCCAAATGGGCAAT
TTTTTCACAGCCATTAGTTTCACCCACGTTAACAGTTCAT
CGGCGGATGTTAACGCCTCGCACAGGGTCTCTCCGCGAAG
AAGTCTTTCGCGCAGTGCACAGCTTATGTTAACATTACTT
TGAATTTTTTGAAACGCTTCATACACATTATGTCCGTATA
ACACTAAATCATCCCATGATACATCGTCATCTGTTGTCGG
TAAACACCCTCCACATTTCCACATCTCGTTTCCACCATTT
ATGACGTTGTGTTTCCTCTGCGGCTCCAGATAGACAGCTC
TCATTTTTTTCATAAACGTTTTCATAAACATTATAAACTT
ACTAATCTCCGTCCCAGACACGTTCAATCCAGCGCTTCTT
ATAGCACGCTGGGTGTTGTGCAGTGGACTCGTACTGTCAG
ATAACTGTTTACGTCTAAACCTCTTGGTCTCCATTTTTGT
TTTCCTTTCCGGTATCGTCAAACACACGCTCAATTCCGCT
TTAACTTTGCCTTTTTACCTCTTGATGCCGGCTGTTGTTA
CTTACATTAAACCCACGAGACTCCTTTTTGTATGTACTGC
GCTCCCCACATATAACTTAAAAGTCTTTTATATGTATGCA
TTTTTCCGTTTTGCATATTTATTATGTGGCCACAACGCCC
ACTTGCTTTCCTTTACCACATCCCTCTACTTGTTTGTAAT
AATCCACTTATGCATTTTTAAACTTTTGCCCTTTTAAAAC
CGCAAACATATACATACATCTTTATACCACATGCGTACCA
AGACATATGTTGTAAGCGCTTTTACGTCTGTTTACATCAC
TGGTTTTTTTTTTTACTACGTGGATTTCAATTCTAACCTT
TTATATATACATGAATTTCCCATTTCTACACTAGCCATTG
TCTAGGTATCCTTTTACCAGTAATAACACATTACTTACGC
ACCGTTTTGCTTACATTGTATGTTACTATTACCATGTCCC
GTAGCCCTACTGTTGAACGCTTCATTTTAAGCTCCACGGA
ATTAACGCTGTCTTCCTTTTCACCCATCACTTACAACGCA
GAATACATGAACGTTTATCTACCGACGTACGAAGAAGCCA
TCCAAGATTTACCGCCTGCATACCGGAGTCGTGAGACATT
ATCAACGCTAACTACAGACACTGGATGTATGGACTGCATT
TGTACAGGCCTGTATAAAATACATGAGCGCTTGACATCAT
GTGTGCGTTATTGTGTCCCGGCCATGTTTGTATTATTCGC
TGTGTTAACCCTAACCGCTGTTATTTTGGTTATATTAGCG
GCGATTCCCGATGTTTAAATAAACCACTGCCATAAATATA
CGCGGTTACTTGTAACGTTTTATTGCCGTTATACTTATTA
GCGCCTATTACTGGGACTTACGCTCTTATATATTAAATAC
ATCACAGCCATTAATGTGCACACTTGTATACATATAAAAA
CAACGTTCCACAACTTTTGCTTATTACCCCCTTTTGTTTT
CCGCCATTCAGGTTTTATGTTTGAGTGCGATGCATTAAAC
AGTGGAATTACAGTTTCTGTTGGGAACACGGTTCTGTTTA
TATATTCGCTCCTTTGGTTATTCATATCACAGGAGTATGT
AAAAACCGAGTCGTCTCCATCCGATTCTTCAGCGTATTTA
CTCCCGCGGGACACCCAGGACATTCCGTGTTTCTTGTATC
CCAGCAATACGTACCGTAGCTTTACTTACTGGTTTTATAT
GTATTTATACACGTCCGTTAATTTATGAATCATCACAAAT
GTGTATGTGAATTTAAAAAATAACTTTAATAAGTCATGTT
CCTTCTTAGTAAATACATGTGTTCATATAATTTATCATGA
TAAATGTAACCAATGCACATTATCCCCTGTTTTTAGCTTA
CTTTACTTTTCTGTACATTATATTCTTTCTTTTCCTTTGT
TGCATATTTAGTCACGGTTTTCTGGTGAAATTCCCATTAC
ATTTCGTACCCGTAACGTACACACGATGTGATCATATGCC
TCCGCTGCACCTGCCCAACAATCAACAGAAAACGGTGTTT
TACAGTGACAATGATATCCTACACCTGATAAATGTACACC
GAGACTTCCGGAATCTAACATTATTTGTAGCGTTGCCGCG
CTAAACGGCGCATCACACAAAGCTGAATAGTTTGCCATTT
CTTTAAGTAAACGCTGACACTGCAATATTTTACAAAGGTT
TTGTATGTCCACAAACAATGGGAATTCGGATGGTTTATCT
TTATGACTTAAGCCAATTAAATATGCGTGCACGTCACCTG
GTTTTGTGCATGTTGCACGTAAAGCACAAAGTCTTTTTGT
TCCATATAACGGCGATGGAGTTGGAGCATGTGAGCTTGTT
GCAGATATCCATGTAGTCTCTAAAGCCAAAGGCGCAACTG
CTTGAGTAAATGGTGCCAGAGCATCGGGTTTTAGGTCTAT
CATTAGCGTTTCCTTATTAACACTTTGGTTACAAGCCAT.

In some embodiments, a nucleic acid sequence fragment of SVV herein can be one or more open reading frame (ORF) sequences of the SVV genome. The SVV genome has 69 potential open reading frames (ORFs) known to date. In accordance with some embodiments, a nucleic acid sequence fragment of SVV herein can be one or more of the 69 ORFs in the SVV genome. In some embodiments, a nucleic acid sequence fragment of SVV herein can be ORFQ, ORFA, ORFB, ORF1, ORF3, or any combination thereof. In some embodiments, a nucleic acid sequence fragment of SVV herein can have a nucleic acid sequence of SVV (e.g., ORFA, ORFB, ORF1, and ORF3) sharing about 85% or more (e.g., about 85%, about 90%, about 95%, about 99%, about 100%) sequence identity to SEQ ID NO: 2.

In some embodiments, chimeric viral constructs herein can have a nucleic acid sequence from any one of the exclusively human pathogenic viruses disclosed herein and any one of the nucleic acid sequence fragments of simian varicella virus (SVV) disclosed herein. In some embodiments, chimeric viral constructs herein can have a nucleic acid sequence from any one of the exclusively human pathogenic viruses disclosed herein and a nucleic acid sequence fragment of simian varicella virus (SVV) disclosed herein, wherein the nucleic acid sequence fragment of SVV can have about 85% (e.g., about 85%, about 90%, about 95%, about 99%, about 100%) or more sequence identity to SEQ ID NO: 1.

In some embodiments, chimeric viral constructs herein can have a nucleic acid sequence from any one of the herpesviruses disclosed herein and any one of the nucleic acid sequence fragments of simian varicella virus (SVV) disclosed herein. In some embodiments, chimeric viral constructs herein can have a nucleic acid sequence from any one of herpesviruses disclosed herein and a nucleic acid sequence fragment of simian varicella virus (SVV) disclosed herein, wherein the nucleic acid sequence fragment of SVV can have about 85% (e.g., about 85%, about 90%, about 95%, about 99%, about 100%) or more sequence identity to SEQ ID NO: 1.

In some embodiments, chimeric viral constructs herein can have a nucleic acid sequence from any one of the VZV sequences disclosed herein and any one of the nucleic acid sequence fragments of simian varicella virus (SVV) disclosed herein. In some embodiments, chimeric viral constructs herein can have a nucleic acid sequence from any one of the VZV sequences disclosed herein and a nucleic acid sequence fragment of simian varicella virus (SVV) disclosed herein, wherein the nucleic acid sequence fragment of SVV can have about 85% (e.g., about 85%, about 90%, about 95%, about 99%, about 100%) or more sequence identity to SEQ ID NO: 1.

In some embodiments, chimeric viral constructs herein can have a nucleic acid sequence of a VZV having one or more stop codons in ORF7 of the VZV genome as disclosed herein and any one of the nucleic acid sequence fragments of simian varicella virus (SVV) disclosed herein. In some embodiments, chimeric viral constructs herein can have a nucleic acid sequence of a VZV having one or more stop codons in ORF7 of the VZV genome as disclosed herein and a nucleic acid sequence fragment of simian varicella virus (SVV) disclosed herein, wherein the nucleic acid sequence fragment of SVV can have about 85% (e.g., about 85%, about 90%, about 95%, about 99%, about 100%) or more sequence identity to SEQ ID NO: 1. In some embodiments, chimeric viral constructs herein can have a nucleic acid sequence of VZV and a nucleic acid sequence fragment of SVV wherein the nucleic acid sequence fragment of SVV can be a truncation of the N-terminal nucleic acid sequence and/or truncation of the C-terminal nucleic acid sequence of SVV.

In certain embodiments, chimeric viral constructs herein can have a nucleic acid sequence of VZV and a nucleic acid sequence fragment of SVV wherein the nucleic acid sequence fragment of SVV is a truncation of the N-terminal nucleic acid sequence fragment of SVV, wherein the N-terminal nucleic acid sequence fragment of SVV can be inserted into one or more locations in nucleic acid sequence of VZV. In some embodiments, an N-terminal nucleic acid sequence fragment of SVV can be inserted at the 5′ end of the nucleic acid sequence of VZV. In some embodiments, an N-terminal nucleic acid sequence fragment of SVV can be inserted at the 3′ end of the nucleic acid sequence of VZV. In some embodiments, an N-terminal nucleic acid sequence fragment of SVV can be inserted between the nucleic acid sequences encoding for ORF 65 and ORF 66 of the of VZV genome. In some embodiments, chimeric viral constructs herein can have a nucleic acid sequence having about 85% (e.g., about 85%, about 90%, about 95%, about 99%, about 100%) or more sequence identity to SEQ ID NO: 3.

In certain embodiments, chimeric viral constructs herein can have a nucleic acid sequence of VZV and a nucleic acid sequence fragment of SVV wherein the nucleic acid sequence fragment of SVV is a truncation of the C-terminal nucleic acid sequence fragment of SVV, wherein the C-terminal nucleic acid sequence fragment of SVV can be inserted into one or more locations in nucleic acid sequence of VZV. In some embodiments, a C-terminal nucleic acid sequence fragment of SVV can be inserted at the 5′ end of the nucleic acid sequence of VZV. In some embodiments, an N-terminal nucleic acid sequence fragment of SVV can be inserted at the 3′ end of the nucleic acid sequence of VZV. In some embodiments, an C-terminal nucleic acid sequence fragment of SVV can be inserted between the nucleic acid sequences encoding for ORF 65 and ORF 66 of the of VZV genome. In some embodiments, chimeric viral constructs herein can have a nucleic acid sequence having about 85% (e.g., about 85%, about 90%, about 95%, about 99%, about 100%) or more sequence identity to SEQ ID NO: 7.

In certain embodiments, chimeric viral constructs herein can have a nucleic acid sequence of VZV and a nucleic acid sequence fragment of SVV wherein the nucleic acid sequence fragment of SVV can be inserted at the 5′ end of the nucleic acid sequence of VZV, at the 3′ end of the nucleic acid sequence of VZV, at any location in the nucleic acid sequence of VZV, or any combination thereof. In some embodiments, chimeric viral constructs herein can have a nucleic acid sequence of VZV and a nucleic acid sequence fragment of SVV wherein the nucleic acid sequence fragment of SVV is inserted at the 5′ end of the nucleic acid sequence of VZV. In some embodiments, chimeric viral constructs herein can have a nucleic acid sequence of VZV and a nucleic acid sequence fragment of SVV wherein the chimeric viral constructs can have a nucleic acid sequence having about 85% (e.g., about 85%, about 90%, about 95%, about 99%, about 100%) or more sequence identity to SEQ ID NO: 4.

In some embodiments, chimeric viral constructs herein can have a nucleic acid sequence of VZV and a nucleic acid sequence fragment of SVV wherein the nucleic acid sequence fragment of SVV is inserted at the 5′ end of the nucleic acid sequence of VZV and the nucleic acid sequence of VZV has one or more stop codons in ORF 7 of the of VZV genome. In some embodiments, chimeric viral constructs herein can have a nucleic acid sequence of VZV and a nucleic acid sequence fragment of SVV wherein the chimeric viral constructs can have a nucleic acid sequence having about 85% (e.g., about 85%, about 90%, about 95%, about 99%, about 100%) or more sequence identity to SEQ ID NO: 6.

In some embodiments, chimeric viral constructs herein can have a nucleic acid sequence of VZV and a nucleic acid sequence fragment of SVV wherein the nucleic acid sequence fragment of SVV is inserted between the nucleic acid sequences encoding for ORF 65 and ORF 66 of the of VZV genome. In other embodiments, chimeric viral constructs herein can have a nucleic acid sequence of VZV and a nucleic acid sequence fragment of SVV wherein the chimeric viral constructs can have a nucleic acid sequence having about 85% (e.g., about 85%, about 90%, about 95%, about 99%, about 100%) or more sequence identity to SEQ ID NO: 3.

In certain embodiments, chimeric viral constructs herein can include a nucleic acid sequence of VZV and a nucleic acid sequence fragment of SVV wherein the nucleic acid sequence fragment of SVV is inserted between the nucleic acid sequences encoding for ORF 65 and ORF 66 of the of VZV genome and the nucleic acid sequence of VZV has one or more stop codons in ORF 7 of the of VZV genome. In other embodiments, chimeric viral constructs herein can include a nucleic acid sequence of VZV and a nucleic acid sequence fragment of SVV wherein the chimeric viral constructs can have a nucleic acid sequence having about 85% (e.g., about 85%, about 90%, about 95%, about 99%, about 100%) or more sequence identity to SEQ ID NO: 4.

Embodiments of the instant disclosure relate methods for generating the chimeric viral constructs disclosed herein. In certain embodiments, any of the chimeric viral constructs disclosed herein can be produced via, e.g., conventional recombinant technology or technology for creating a chimera. In some examples, nucleic acid sequences disclosed herein can be readily isolated and sequenced using conventional procedures (e.g., by using oligonucleotide probes that are capable of binding specifically to genes encoding a polypeptide sequence). Once isolated, the nucleic acid sequences herein can be placed into one or more expression vectors, which can then be transfected into host cells such as E. coli cells, simian COS cells, Chinese hamster ovary (CHO) cells, Human Embryotic Kidney (HEK) 293 cells or myeloma cells that do not otherwise produce the proteins, protein domains, peptides, peptide fragments, and/or polypeptides encoded by the nucleic acid sequences disclosed herein. In some embodiments, nucleic acid sequences can then be modified accordingly for generating any of the compositions disclosed herein.

In certain embodiments, nucleic acid sequences disclosed herein can be cloned into an expression vector, each nucleotide sequence being in operable linkage to a suitable promoter. In other embodiments, each of the nucleic acid sequences disclosed herein can be in operable linkage to a distinct prompter. Alternatively, nucleic acid sequences disclosed herein can be in operable linkage with a single promoter, such that one or more proteins are expressed from the same promoter. In some embodiments, an internal ribosomal entry site (IRES) can be inserted between protein encoding sequences of the nucleic acid sequences disclosed herein.

In some embodiments, nucleic acid sequences disclosed herein can be cloned into two vectors, which can be introduced into the same or different cells. In other embodiments, any of the proteins, protein domains, peptides, peptide fragments, and polypeptides as encoded by nucleic acid sequences disclosed herein can be expressed in different cells, each of them can be isolated from the host cells expressing such and the isolated proteins can be mixed and incubated under suitable conditions allowing, for example, methods of detecting protein interactions as disclosed herein.

In certain embodiments, nucleic acid sequences disclosed herein can be cloned into a suitable expression vector in operable linkage with a suitable promoter using methods known in the art. In certain embodiments, nucleic acid sequences disclosed herein including a vector can be contacted, under suitable conditions, with a restriction enzyme to create complementary ends on each molecule that can pair with each other and be joined together with a ligase. In some embodiments, synthetic nucleic acid linkers can be ligated to the termini of a gene. In some embodiments, these synthetic linkers can contain nucleic acid sequences that correspond to a particular restriction site in the vector. One or skill the art can appreciate that selection of expression vectors/promoter would depend on the type of host cells for use in producing the decoy fusion proteins.

In certain embodiments, a variety of promoters can be used for any of nucleic acid sequences disclosed herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter. In some embodiments, regulatable promoters can also be used. Such regulatable promoters can include, but are not limited to, those using the lac repressor from E. coli as a transcription modulator to regulate transcription from lac operator-bearing mammalian cell promoters and those using the tetracycline repressor (tetR). Other systems suitable for use herein can include FK506 dimer, VP16 or p65 using astradiol, RU486, diphenol murislerone, or rapamycin.

In other embodiments, vectors used herein can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

In certain embodiments, one or more vectors (e.g., expression vectors) encompassing any of the nucleic acid sequences disclosed herein can be introduced into suitable host cells for producing the any of the proteins, protein domains, peptides, peptide fragments, or polypeptides encoded by the nucleic acid sequences herein. In some embodiments, host cells herein can be cultured under suitable conditions for expression of any of the proteins, protein domains, peptides, peptide fragments, and polypeptides encoded by the nucleic acid sequences herein. Such proteins, protein domains, peptides, peptide fragments, and/or polypeptides encoded by the nucleic acid sequences herein can be recovered by the cultured cells (e.g., from the cells or the culture supernatant) via a conventional method, e.g., affinity purification. If necessary, any of the proteins, protein domains, peptides, peptide fragments, or polypeptides encoded by the nucleic acid sequences herein can be incubated under suitable conditions for a suitable period of time allowing for production of the proteins, protein domains, peptides, peptide fragments.

In some embodiments, methods for preparing any of the nucleic acid sequences disclosed herein described herein can involve a recombinant expression vector that encodes all components of the any of nucleic acid sequences the herein. In some embodiments, recombinant expression vectors can be introduced into a suitable host cell (e.g., a HEK293T cell or a dhfr-CHO cell) by a conventional method, e.g., calcium phosphate-mediated transfection. In some embodiments, positive transformant host cells can be selected and cultured under suitable conditions allowing for the expression of any of the proteins, protein domains, peptides, peptide fragments, and polypeptide encoded by nucleic acid sequences disclosed herein which can be recovered from the cells or from the culture medium.

In certain embodiments, any of the chimeric viral constructs herein can be formulated into a pharmaceutical composition. In some embodiments, a pharmaceutical composition including any of the chimeric viral constructs herein can further include a pharmaceutically acceptable carrier, diluent, excipient, or any combination thereof of use for delivering the constructs to a host or storing the constructs. As used herein “pharmaceutically acceptable” refers to molecular entities and other ingredients of compositions that are physiologically tolerable and do not typically produce untoward reactions when administered to a mammal (e.g., a non-human primate model, a human). In some embodiments, pharmaceutically acceptable carriers can include, but are not limited to phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

In some embodiments, the pharmaceutical compositions or formulations herein can be used for parenteral administration, such as intravenous, intracerebroventricular injection, intra-cisterna magna injection, intra-parenchymal injection, or a combination thereof. In some embodiments, the pharmaceutical compositions or formulations herein can be used for subcutaneous injection. In accordance with some embodiments, pharmaceutically acceptable carriers herein can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein can further include additional ingredients, for example preservatives, buffers, tonicity agents, antioxidants and stabilizers, nonionic wetting or clarifying agents, viscosity-increasing agents, and the like. In some embodiments, pharmaceutical compositions described herein can be packaged in single unit dosages or in multi-dosage forms. In some embodiments, pharmaceutical compositions described herein can be packaged as a kit.

In some embodiments, formulations suitable for parenteral administration herein can include aqueous and non-aqueous sterile injection solutions which can include anti-oxidants, buffers, bacteriostats and solutes which render the formulation isotonic with the blood of the intended recipient; and aqueous and non-aqueous sterile suspensions which may include suspending agents and thickening agents. In some embodiments, aqueous solutions for use herein be suitably buffered (preferably to a pH of from 3 to 9).

Embodiments of the instant disclosure relate to compositions and methods for creating a non-human animal model (e.g., a non-human primate model). In some embodiments, the animal models disclosed herein can be non-human animal models. Non-limiting examples of non-human animal models can include rabbits, rodents (e.g., mice, rats, hamsters, gerbils, and guinea pigs), cows, sheep, pigs, goats, horses, dogs, cats, birds (e.g., chickens, turkeys, ducks, geese), non-human primates (NHP), and the like. In certain embodiments, the animal models disclosed herein can be non-human primate models. In some embodiments, non-human primate models herein can be a rhesus macaque (Macaca mulatta), a cynomolgus macaque (Macaca fascicularis), an African green monkey (Chlorocebus sabaeus), a baboon species from the genus Papio (e.g., a hamadryas baboon, a Guinea baboon, a olive baboon, a yellow baboon, a chacma baboon), a sooty mangabey (Cercocebus atys), a common marmoset (Callithrix jacchus), a capuchin of the genus Cebus, an owl monkey (Aotus trivirgatus), a patas money (Erythrocebus patas), a pigtail macaque (Macaca nemestrina), a sabaeus monkey (Chlorocebus sabaeus), a squirrel monkey of the genus Saimiri (e.g., S. Cebidae, S. Platyrrhini), a tamarin of the genus Saguinus, or any combination thereof.

In some embodiments, non-human primate models herein can be used to study at least one exclusively human pathogenic virus of interest and/or test one or more antiviral agents against one or more exclusively human pathogenic virus of interest. In some embodiments, the exclusively human pathogenic virus of interest can be an RNA virus. As used herein, an “RNA virus” refers to a virus that has an RNA genome. In some embodiments, non-human primate models can used to study or test antiviral agents against an RNA virus of interest from the virus family Reoviridae, Picornaviridae, Caliciviridae, Togaviridae, Arenaviridae, Flaviviridae, Orthomyxoviridae, Paramyxoviridae, Bunyaviridae, Rhabdoviridae, Filoviridae, Coronaviridae, Astroviridae, Bornaviridae, Arteriviridae, Hepeviridae, or any combination thereof. In some embodiments, the exclusively human pathogenic virus of interest can be can be a DNA virus. As used herein, a “DNA virus” refers to viruses that have a DNA genome. In some embodiments, the human DNA virus of interest can be from the virus family Adenoviridae, Papovaviridae, Parvoviridae, Herpesviridae, Poxviridae, Anelloviridae, Pleolipoviridae, or any combination thereof. In some embodiments, the exclusively human pathogenic virus of interest can be a retrovirus (e.g., HIV, HTLV, and Lenti), a picoma virus (e.g., polio), a flavirus (e.g., HCV and other pestiviruses such as BVDV), a plant virus (e.g., capilloviruses), a togoviruses (e.g., sindbis), a parvo virus, a coronavirus, (e.g HCoV -229E, HCoV-0C43, HCoV-NL63, HCoV-HKU1, Severe Acute Respiratory Syndrome Coronavirus (SARS-CoV), MERS-CoV (Middle East Respiratory Syndrome Coronavirus), and SARS-CoV-2 (Severe Acute Respiratory Syndrome Coronavirus-2 otherwise known as COVID-19)), a viral hemorrhagic fevers (VHFs) (e.g., Ebola, Marburg, Lassa fever, and yellow fever), and the like.

In some embodiments, the exclusively human pathogenic virus of interest can be a herpes virus. In some embodiments, non-human primate models can be used to study or test an antiviral agent mimic at least one human virus of interest wherein the human virus of interest can be human herpes simplex virus type 1 (HSV-1), herpes simplex virus type 2 (HSV-2), Epstein-Barr-virus (EBV), cytomegalovirus (CMV), human herpes virus type 6 (HHV-6), human herpes virus type 7 (HHV-7), human herpes virus type-8 (HHV-8), varicella zoster virus (VZV), or any combination thereof.

In some embodiments, non-human primate models disclosed herein can be used to assess characteristics or side effects presented by a human infected with the exclusively human pathogenic virus of interest. In some embodiments, a characteristic presented by a human infected with the virus of interest can be a clinical symptom, a pathological symptom, a cognitive symptom, or any combination thereof. One of skill in the art can appreciate that a clinical symptom, a pathological symptom, a cognitive symptom, or any combination thereof can depend on the virus at the source of the infection. Non-limiting examples of clinical symptoms of human viral infection can include sneezing, nasal discharge and obstruction, sore throat, coughing, muscle pains, malaise and mood changes, fever, febrile seizures, diarrhea, nausea, vomiting, rash, or other dermal disturbances, swollen tonsils, swollen lymph nodes, severe lung infection, neuronal damage, balance issues, visual side effects and the like. Non-limiting examples of pathological symptoms of human viral infection can include viral titer, viral latency, viral spread in one or more tissues, viral accumulation in one or more tissues, cytokine production, chemokine production, tissue inflammation, vascular modifications, modification of the immune system, cellular apoptosis, and the like. Non-limiting examples of cognitive symptoms of human viral infection can include stroke, changes in mood, changes in memory, changes in motor function (e.g., paralysis, modified gait), neuroinflammation, vasculopathy, myelitis, zoster paresis, and the like. In some embodiments, a non-human primate models herein can mimic at least 85% (e.g., about 85%, 90%, 95%, 99%, 100%) of the characteristics presented by a human infected with the virus of interest.

In some embodiments, non-human primate models herein can be used to screen for in vivo effects of a vaccine. As used herein, the term “vaccine” refers to any compound/agent (“vaccine component”), or combinations thereof, capable of inducing/eliciting an immune response in a host (an immunogenic agent) and which permits treatment, amelioration and/or prevention of an infection and/or a disease. Non-limiting examples of such antiviral agents include proteins, polypeptides, protein/polypeptide fragments, immunogens, antigens, peptide epitopes, epitopes, mixtures of proteins, peptides or epitopes as well as nucleic acids, genes or portions of genes (encoding a polypeptide or protein of interest or a fragment thereof) added separately or in a contiguous sequence such as in nucleic acid vaccines, and the like. Non-limiting examples of in vivo effects of a vaccine can include reduction in viral titer, reduction in viral shedding, reduction of storage of latent virus in a cell or tissue, modification/prevention/amelioration of reactivation of a latent virus stored in a cell or tissue, modifies viral inflammation in a cell or tissue, modification of one or more characteristics (e.g., a clinical symptom, a pathological symptom, a cognitive symptom) presented by a human infected with the virus of interest.

In some embodiments, compositions and methods disclosed here can be used for screening new antiviral candidates in vivo. As used herein, an “antiviral drug” can mean a biomolecule that decreases viral function or viral titer or side effects of viral infection. In some embodiments, non-human primate models herein can be used to screen for efficacy of an antiviral drug that targets any one of the exclusively human pathogenic viruses disclosed herein. In some embodiments, non-human primate models herein can be used to screen for an antiviral drug that targets herpesviruses.

In some embodiments, non-human primate models herein can be used to screen for efficacy of an antiviral drug or antiviral agent that is not currently known or has been used for treatment of other viruses but not known to treat a specific virus such as an exclusively human pathogenic virus disclosed herein. In other embodiments, non-human primate models herein can be used to screen for physiological effects of an antiviral drug that is currently known. Non-limiting examples of currently known antiviral drugs suitable for use to assess efficacy against a target virus can include acyclovir, brivudin, cidofovir, famciclovir, fomivirsen, foscarnet, ganciclovir, penciclovir, valacyclovir, valganciclovir, vidarabine, amantadine, rimantadine, oseltamivir, zanamivir, interferons (IFNs), ribavirin, adefovir, emtricitabine, entecavir, lamivudine, telbivudine, tenofovir, boceprevir, telaprevir, famcyclovir, alpha-methylbenzyl thiourea derivatives, pyrazolo derivatives, WAY-150183, chlorobenzothiphen derivatives, and the like.

In some embodiments, in vivo screening systems herein can include any one of the non-human primate models herein. In some embodiments, in vivo screening systems herein can further include at least one assay capable of measuring at least one positive output, at least one negative output, or any combination thereof from the in vivo screening system. As used herein, an “output” can be a measurement of one or more characteristics or side effects known to present in a human infected with an exclusively human pathogenic virus of interest. In some embodiments, an “output” can be a measurement of a clinical symptom, a pathological symptom, a cognitive symptom, or any combination thereof as disclosed herein. A “positive output” herein can be an attenuation, a prevention, a reversal or any combination thereof of the one or more characteristics or side effects known to present in a human infected with an exclusively human pathogenic virus of interest. A “negative output” herein can be an exacerbation, an increase, an appearance or any combination thereof of the one or more characteristics or side effects known to present in a human infected with an exclusively human pathogenic virus of interest. Non-limiting examples of assays that can be used herein for measuring at least one positive output, at least one negative output, or any combination thereof can include: blood chemistries, flow cytometry for one or more immune cell types, ELISA assays, western blotting, RT-PCR, tests for cognitive performance, imagining procedures (e.g., Mill, PET, CT), immunohistochemistry, genetic screenings, standard clinical observation methodologies and the like.

In certain embodiments, the present disclosure provides methods of making any one of the non-human primate models disclosed herein. In certain embodiments herein, methods of making non-human primate models disclosed herein can include infecting a non-human primate with any one of the chimeric viral constructs disclosed herein. In some embodiments herein, methods of infecting a non-human primate can include inoculating a non-human primate intravenously (i.v.) with a chimeric virus generated from any one of the chimeric viral constructs disclosed herein.

In certain embodiments, the present disclosure provides gene therapy delivery systems and methods of use. In some embodiments, a gene therapy delivery system herein can include at least one vector, wherein the vector includes a nucleic acid sequence of open reading frame 7 (ORF7) of human VZV. In some embodiments, a nucleic acid sequence of ORF7 of human VZV for use in gene therapy delivery systems can share about 85% or more (e.g., about 85%, 90%, 95%, 99%, 100%) sequence identity to SEQ ID NO: 8. In some embodiments, a nucleic acid sequence of ORF7 of human VZV for use in gene therapy delivery systems can encode for a protein which can share about 85% or more (e.g., about 85%, 90%, 95%, 99%, 100%) sequence identity to SEQ ID NO: 9.

In some embodiments, a vector for use in gene therapy delivery systems herein can be a viral vector, a non-viral vector, or any combination thereof. Non-limiting examples of viral vectors suitable for used herein can include a retrovirus, an adenovirus, an adeno-associated virus, a lentivirus, a pox virus, an alphavirus, a herpes virus, or any combination thereof. Non-limiting examples of non-viral vectors suitable for used herein can include naked DNA, an oligonucleotide, a polynucleotide, a lipoplex, a polyplex, or any combination thereof.

In some embodiments, gene therapy delivery systems herein can be used to deliver one or more agents to a target tissue. In some embodiments, gene therapy delivery systems herein can be used to deliver one or more agents to neuronal tissue, skin, or a combination thereof. In some embodiments, gene therapy delivery systems herein can be used to deliver one or more agents across the blood-brain-barrier. In some embodiments, gene therapy delivery systems herein can be used to deliver one or more agents to ganglia.

In some embodiments, a nucleic acid sequence of open reading frame 7 (ORF7) of human VZV for use in gene therapy delivery systems can be modified at one or more nucleic acids. In some embodiments, a nucleic acid sequence of open reading frame 7 (ORF7) of human VZV for use in gene therapy delivery systems can be modified at one or more nucleic acids to introduce one or more stop codons. In some embodiments, a nucleic acid sequence of open reading frame 7 (ORF7) of human VZV for use in gene therapy delivery systems can share about 85% (e.g., about 85%, 90%, 95%, 99%, 100%) or more sequence identity to SEQ ID NO: 8.

In some embodiments, gene therapy delivery systems herein can be used in methods of treating a disease or a condition in a subject. As used herein, the term “treating” can refer to the application or administration of a gene therapy delivery system herein to a subject, who is in need of the treatment, for example, having a target disease or condition, a symptom of the disease/condition, or a predisposition toward the disease/condition, with the purpose to cure, heal, alleviate, relieve, alter, remedy, ameliorate, improve, or affect the disorder, the symptom of the disease, or the predisposition toward the disease or disorder. Non-limiting examples of diseases or conditions to be treated with gene therapy delivery systems herein can be viral infections, neurological disorders (e.g., Alzheimer's disease, Parkinson's disease, amyotrophic lateral sclerosis, and the like), dermatological disorders (e.g., genetic diseases, skin cancer, chronic wound, intractable inflammatory disease, and the like), or any combination thereof.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, the term “subject,” “subject recipient” and “patient” are used interchangeably herein and refer to human subjects. The term “non-human animals” of the disclosure includes all vertebrates, e.g., mammals and non-mammals, such as nonhuman primates, sheep, dog, cat, horse, cow, chickens, amphibians, reptiles, and the like. In some embodiments, the subject can be a human such as an adult, child, adolescent or infant.

As used herein, “treatment,” “therapy” “treatment regimen” and/or “therapy regimen” can refer to the clinical intervention made in response to a disease, disorder or physiological condition manifested by a subject or to which a subject can be susceptible. The aim of treatment includes the alleviation or prevention of symptoms, slowing or stopping the progression or worsening of a disease, disorder, or condition and/or the remission of the disease, disorder or condition. As used herein, the term “treating” can refer to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

As used herein, “prevent” or “prevention” refers to eliminating or delaying the onset of a condition, disorder, disease or physiological condition, or to the reduction of the degree of severity of a condition, disorder, disease or physiological condition, relative to the time and/or degree of onset or severity in the absence of intervention.

The term “effective amount” or “therapeutically effective amount” refers to an amount sufficient to effect beneficial or desirable biological and/or clinical results.

Alleviating a target disease/disorder or condition includes delaying the development or progression of the disease, or reducing disease severity or prolonging survival. Alleviating the disease or prolonging survival does not necessarily require curative results. As used therein, “delaying” the development of a target disease, condition or disorder can mean to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

The term “biomolecule” as used herein refers to, but is not limited to, proteins, enzymes, antibodies, DNA, siRNA, and small molecules. “Small molecules” as used herein can refer to chemicals, compounds, drugs, and the like.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide,” and “protein” are used interchangeably, and refer to a compound having amino acid residues covalently linked by peptide bonds. A protein or peptide must contain at least two amino acids, and no limitation is placed on the maximum number of amino acids that can have a protein's or peptide's sequence. Polypeptides include any peptide or protein having two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “encode” is used herein to refer to the capacity of a nucleic acid to serve as a template for transcription of RNA or the capacity of a nucleic acid to be translated to yield a polypeptide. Thus a DNA sequence that is transcribed to yield an RNA is said to “encode” the RNA. If a nucleic acid sequence is transcribed to yield an RNA that is translated to yield a polypeptide, both the nucleic acid and the RNA are said to encode the polypeptide. “Transcription” as used herein includes reverse transcription, where appropriate.

The term “biological sample” can include a sample obtained from subject (e.g., a bodily fluid, a tissue). Non-limiting biological samples obtained from subject suitable for use herein can include blood, sputum, plasma, serum, cell scrapings, tissues, biopsies, teeth, perspiration, fingernail, skin, hair, feces, urine, semen, mucus, saliva, gastrointestinal tract samples, and the like.

Unless otherwise defined, all technical terms used herein have the same meaning as commonly understood by one of ordinary skill in the art to which this disclosure belongs.

The following constructs, chimeric viral constructs, primers, and nucleic acid sequences provided in Table 1 are included to illustrate certain embodiments. It should be understood by one of skill in the relevant art that that any of the following sequences can be modified in accordance with the present disclosure.

TABLE 1

| | |
|---|---|
| Name | SEQ ID NO |

Inserts from SVV

| | |
|---|---|
| SVV Left end ORF3 (—N) | 1 |
| SVV ORFsA-3 (—NN) | 2 |

Complete constructs

| | |
|---|---|
| SVZV-NN/LE-NN Construct | 3 |
| SVZV-N Construct | 4 |
| SVZV-NN-D7 construct | 5 |
| SVZV-N-D7 construct | 6 |
| SVZV-RE-NN construct | 7 |

ORF7

| | |
|---|---|
| ORF7 WT nucleotide | 8 |
| ORF7 WT protein | 9 |
| ORF7 Mutant nucleotide (just first line) | 10 |
| (TAGCTGACTAAGTGTGCCAGCTTATGTGGATATG) | |

Primers

| | |
|---|---|
| ORFA-3 forward primer (—NN) | 11 |
| ORFA-3 reverse primer (—NN) | 12 |
| Left end ORF3 forward primer (—N) | 13 |
| Left end ORF3 reverse primer (—N) | 14 |
| ORF7-D7 forward primer | 15 |
| ORF7-D7 reverse primer | 16 |

Full Genomes

| | |
|---|---|
| VZV Genome | 17 |
| SVV Genome | 18 |

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1

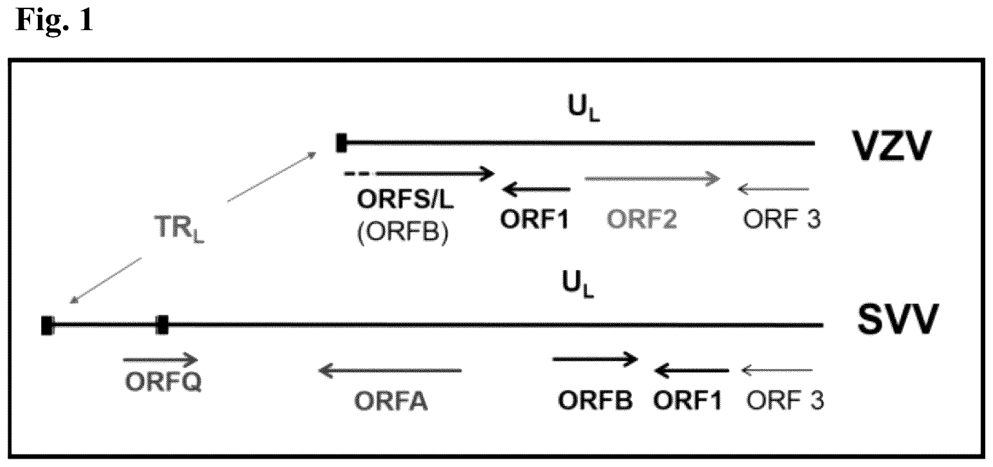

In one exemplary method, varicella-zoster virus (VZV) infectivity was analyzed. In accordance with this example, inoculation of VZV into mice, guinea pigs, and rats do not mimic VZV infection or latency in humans. It is noted that simian varicella virus (SVV) infection in non-human primates (NHP) can mimic human VZV infection but there is currently no way to study VZV infection in non-human primates directly. Further, the mechanism of VZV neuroattenuation in human is not completely understood, in part due to the discrepancy in results obtained from nonhuman model when compared with the human studies. While VZV and SVV genomes are similar, they have some differences, as illustrated in FIG. 1, at the left end of their genomes. FIG. 1 illustrates a partial genomic organization at the left end of VZV and SVV genomes. As illustrated in FIG. 1, the VZV and SVV viral genomes are very similar except for the open reading frames (ORFs) ORF A and ORF Q (present in SVV and not VZV) as well as ORF2 (present in VZV but not in SVV). As illustrated in FIG. 1, the terminal repeat sequences (TRL), and left most part of the unique long sequences (UL) are indicated. In addition, it is noted that SVV ORF B is homologous to VZV ORF S/L. The localized differences was studied and chimeric constructs referenced as SVZVs are illustrated as described below.

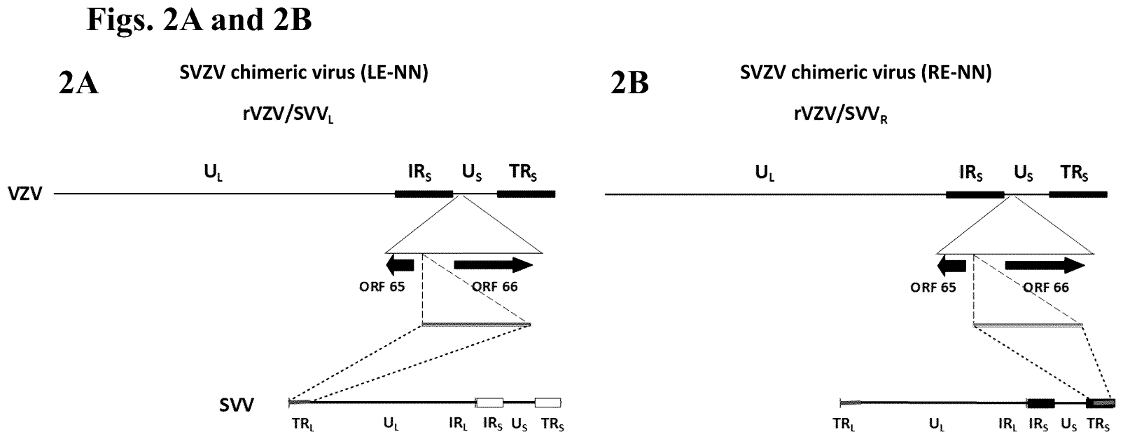

In certain exemplary methods, two recombinant SVZV chimeric viruses, SVZV-LE-NN and SVZV-RE-NN (FIGS. 2A and 2B), were constructed by inserting sequences located at the left (SVV-LE-NN; SEQ ID NO: 3) and right end (SVV-RE-NN; SEQ ID NO: 7) of the SVV genome, respectively, between VZV ORFs 65 and 66 (non-native site) using homologous recombination of overlapping cosmids. As demonstrated, SVZVs contained the complete VZV genome with a unique ˜3 kb SVV segment as an insert. SVZV-LE-NN, but not SVZV-RE-NN, replicated in culture and was infectious in vivo (see Example 2).

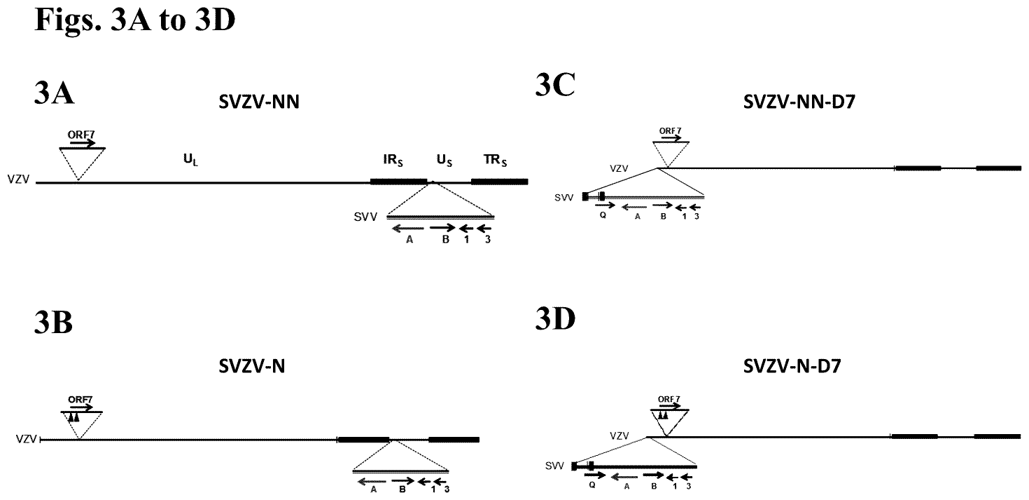

As demonstrated, bacterial artificial chromosome (BAC) technology was used to generate recombinant SVZV chimeric viruses (FIGS. 3A to 3D). An example of the generation of recombinant BACs is described herein.

It was hypothesized that the insertion of SVV-specific sequences into the non-native site (SVZV-NN) could disrupt the organization of the VZV genome, leading to the loss of some pathogenic functions, and interrupt important VZV regulatory elements in this region. For example, alpha herpes virus left end sequences are important in cleavage and packaging and are important in virus spread into multiple tissues. Accordingly, an additional recombinant virus, SVZV-N, was generated by inserting the SVV sequences into its native left-end location on the VZV genome (FIG. 3C, Table 1). BAC technology using a galK selection scheme was used to generate the recombinant SVZVs. A BAC is a DNA construct, based on a functional fertility plasmid (or F-plasmid), used for transforming and cloning in bacteria. The galK selection scheme is generally a two-step system: the first step, a positive selection step, involves targeting the region of interest with the galK cassette containing homology to a specified position in a BAC. The recombinant bacteria are selected on the minimal plate containing galactose as the carbon source. In the second step, a DNA fragment containing the particular mutation of interest replaces the galK cassette. Cells are selected for the loss of galK in the presence of 2-deoxy-galactose (DOG) on minimal plates with glycerol as the carbon source.

To generate the recombinant SVZVs herein, a BAC recombineering (recombination-mediated genetic engineering) method was used to first insert galk cassette at the desired locations in the VZV genome shown in Table 2. Briefly, SVZV-NN and SVZV-N were prepared using primers designed to amplify the galK gene from plasmid pGalK flanked by VZV sequences (sequences between ORF65 and 66 for SVZV-NN and ORF1 and 3 for SVZV-N). This cassette was used to transform electrocompetent SW102 E. coli containing VZV rOka BAC. Following homologous recombination, the galK was inserted between ORF65 and 66 or replacing the left end of VZV containing ORFs 1-3. Results were validated by testing for galK-conferred growth, PCR and sequencing.

TABLE 2

| | | SVV insert | | |
|---|---|---|---|---|
| | VZV | Location of fragment | Size | Location on |
| SVZV | ORF7 | on SVV genome | (bp) | VZV genome |
| NN | Wild | ORFs A-3 | 2,955 | Avr II site at |
| | type | (645-3,600) | | 112,855 |
| NN-7D | Mutant | | | |
| N | Wild | Left terminus-ORF3 | 3,600 | Replaces nucleotides |
| | type | (1-3,600) | | 1-2,460 |
| N-7D | Mutant | | | at the left end |

In the next step, the galK cassette was replaced with SVV sequences. Specifically, to generate a SVZV-NN construct (SEQ ID NO: 3 (SVZV-NN construct)), SVV ORFsA-3 (645-3,600 bp, SEQ ID NO: 2) was inserted at 112,855 bp in the VZV genome without any deletion (SEQ ID NO: 17 (VZV genome)). To generate SVZV-N (SEQ ID NO: 4 (SVZV-N construct)), 1-2,400 bp located at the left end of the VZV genome was replaced with SVV left end-ORF3 (1-3,600 bp, SEQ ID NO: 5). The 3-kb SVV ORFA-3 sequence (SEQ ID NO: 2) or SVV left end-ORF3 (SEQ ID NO: 1) were PCR-amplified from SVV BAC with primers flanking the galK gene and high-fidelity long-range PCR Kit. Primers for amplifying the 3-kb SVV ORFA-3 sequence from SVV BAC for the SVZV-NN construct were: SEQ ID NO: 11 and SEQ ID NO: 12. Primers used for amplifying SVV left end-ORF3 (1-3,600bp, SEQ ID NO: 1) from SVV BAC for the SVZV-N construct were: SEQ ID NO: 13 and SEQ ID NO: 14.

Electroporation of the product into E. coli SW102 harboring VZV-galK BACs and activation of the recombination system results in the chimeric SVZV BAC DNA, as verified by the counter-selection against galK with 2-deoxygalactose minimal media agar, PCR analysis and DNA sequencing.

Table 3 lists putative functions of ORFs at the left end of VZV and SVV genomes and their HSV-1 orthologues.

TABLE 3

| ORF | | | | Growth | |
|---|---|---|---|---|---|
| VZV | SVV | Structural features/ | HSV-1 | Dependence | |
| (bp) | (bp) | putative function | orthologue | VZV | SVV |
| | Q (417) | Unknown | None | Absent | Unknown |
| S/L (0) | B (344) | Type 2 transmembrane | UL56 | Essential | Unknown |
| (390) | | protein interacting with | | | |
| | | kinesin motor protein | | | |
| | A (881) | Homologous to the C-terminus | UL54 | Absent | Nonessential |
| | | of VZV and SVV immediate- | | | |
| | | early transactivator 4 protein | | | |
| 1 (327) | 1 (298) | Transmembrane protein | None | Nonessential | Unknown |
| 2 (717) | | Membrane phosphoprotein | None | Nonessential | Absent |
| | 3 (488) | Unknown | UL55 | Nonessential | Unknown |

VZV ORF7 (SEQ ID NO: 8), a neurotropic factor, has is a 93% identity to SVV-ORF7 protein. As such, recombinant viruses having the SVV-specific inserts described herein were also modified to add three stop codons within VZV ORF7, thus allowing for specific deletion of that reading frame and analysis of ORF7 function (see Example 3). SVZV constructs SVZV-NN-7D and SVZV-N-7D were prepared in the same manner as SVZV-NN and SVZV-N, respectively, except for introduction of three stop codons within VZV ORF7 (5′-TAGCTGACTAAGTGTGCCAGCTTATGTGGATATG-3; SEQ ID NO: 10).

The four constructs: SVZV-NN, SVZV-N, SVZV-NN-7D, and SVZV-N-7D are shown in FIGS. 3A to 3D and described in Table 2 herein.

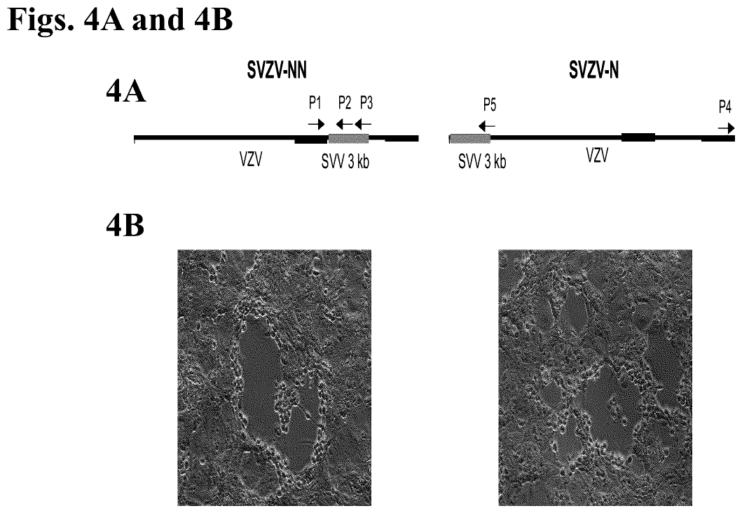

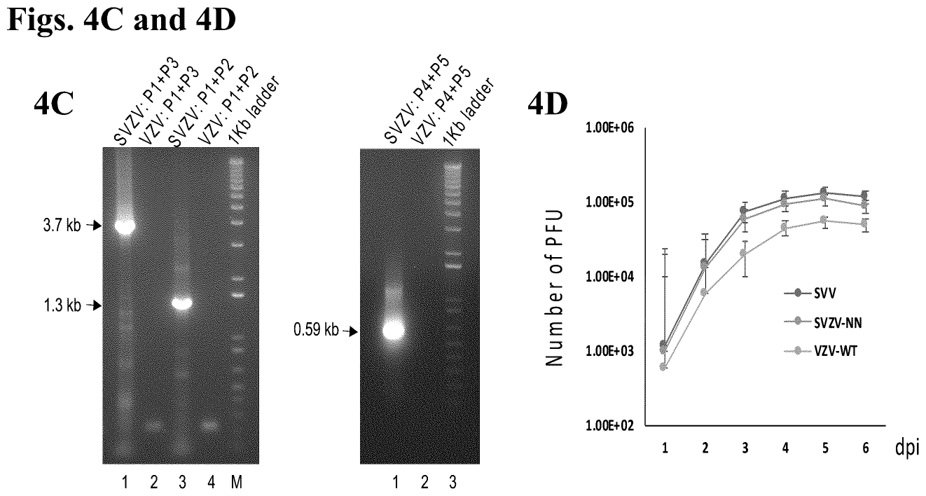

In an exemplary proof of concept, when the two recombinant SVZV DNAs without mutations in ORF7 (e.g., illustrated in FIG. 3A, FIG. 3C and FIG. 4A) were transfected into Vero cells, virus plaques were seen suggesting that insertion of foreign DNA did not alter the viability of the virus (FIG. 4B). The identity of the recombinant viruses was validated extensively by PCR analysis (FIG. 4C). To test the SVZV growth kinetics, Vero cells were infected with VZV, SVV and SVZV-NN at MOI of 0.1. SVZV-NN were demonstrated to grow in the Vero cells comparable to SVV and VZV (FIG. 4D). This result also demonstrated that SVZV-NN has retained its packaging signal.

In other methods, the chimeric viruses produced as described herein were validated to confirm stability. Specifically, the inserted target DNA in 5-6 independent chimeric BAC clones was sequenced to confirm the absence of any mutation. Spe I-digested samples BAC clones and WT VZV were examined by agarose gel electrophoresis. Recombinant BAC DNA's containing SVZV-NN-D7 and SVZV-N-D7 were co-transfected into Vero cells with a Cre-expression plasmid to remove the BAC vector from the viral genome. SVZVs were characterized by growth curve analysis in rhesus fibroblasts and human retinal pigment epithelial (ARPE-19) cells, and by whole-genome sequencing (GENEWIZ, NJ) to eliminate any additional mutations. Stocks of the four chimeric SVZV (1×10⁶ PFU) were generated for inoculation into NHP. The growth curve and pathogenesis of SVZVs was analyzed as described below in Examples 4 to 5.

### Example 2

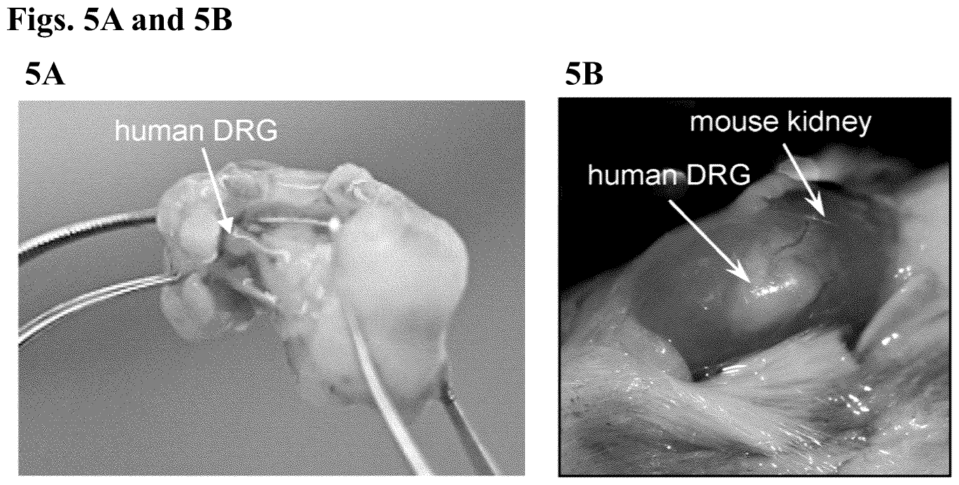

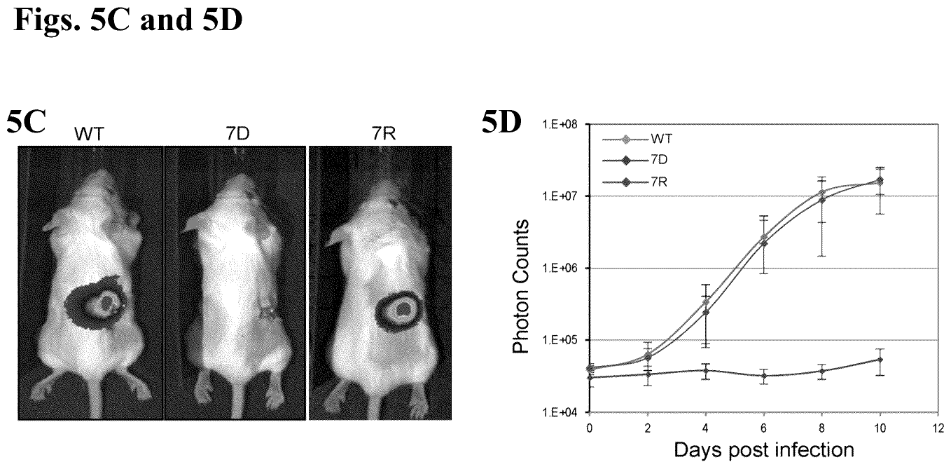

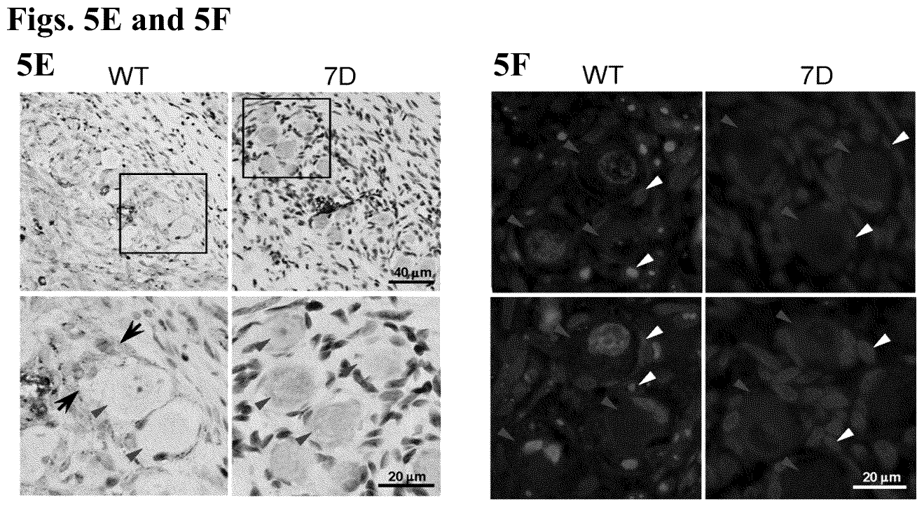

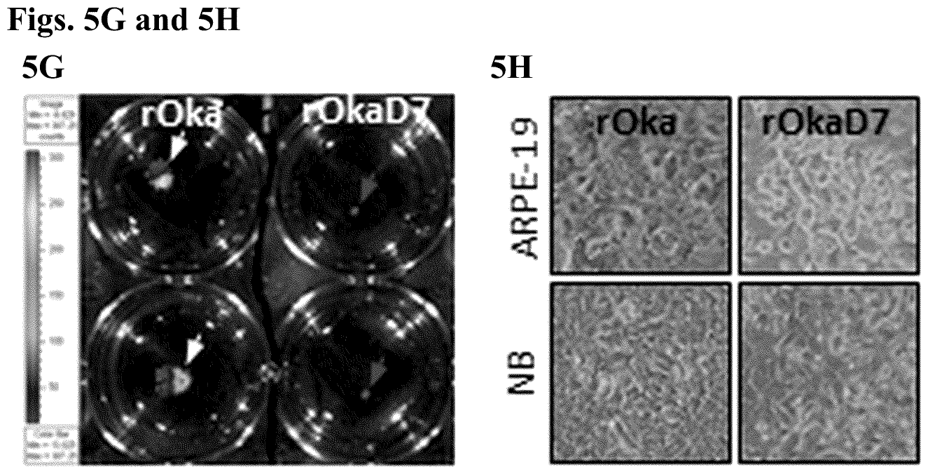

In another exemplary method, human fetal dorsal root ganglion (DRG) and skin samples (˜20 weeks gestational age) were obtained to use in several experiments for testing infectivity. DRG and the human fetal skin were implanted under the kidney capsule and under the skin of C.B-17 SCID/SCID mice, respectively, as an acceptable model. Four weeks after implantation, the implanted tissues were surgically exposed for access and inoculated directly with 100 PFU of VZVs expressing luciferase. Luciferase activity within the implants are measured every 48 hours for 10 days as above. Human DRG xenografts were inoculated in SCID mice with rOka (or WT), rOka lacking ORF7 (rOkaD7 or 7D), and rOka with a replaced WT ORF7 (7R) where “rOka” was a recombinant varicella-zoster virus (VZV) Oka vaccine strain. FIGS. 5A and 5B illustrate representative images of the under-kidney location of the DRG xenografts in these mice. Virus growth was measured by in vivo imaging systems (IVIS) 10 days post inoculation (dpi) and a representative image of the mice examined is provided in FIG. 5C and a growth curve of the three viruses in the implant is provided in FIG. 5D. Immunohistochemistry (IHC) and fluorescence in situ hybridization (FISH) staining of VZV-infected DRG were performed at 10 dpi. IHC was performed using anti-VZV gE and representative images of WT and 7D infected mice are shown in FIG. 5E where red arrowheads indicate DRG neurons. Extensive cytopathic effects and apoptotic aggregates (white arrowheads) were observed in WT-infected DRG samples, correlating with VZV replication and the presence of viral gE antigen (brown). In contrast, 7D infected DRG cells exhibited normal morphology with rounded cell bodies in the absence of VZV gE. Fluorescent in situ hybridization (FISH) staining using a VZV DNA probe demonstrated similar results. VZV DNA was detected in the intranuclear replication centers in WT-infected neurons (green), but not in 7D-infected neurons (FIG. 5F, neurons (red arrowheads) and satellite cells (white arrowheads) are indicated). Fetal DRG were cultured ex vivo to determine whether rOkD7 could expand. As illustrated in FIG. 5G, rOkaD7 (7D) failed to expand in ex vivo DRG culture as examined using IVIS (9 dpi). Further, as illustrated in FIG. 5H, rOkaD7 (7D) infects failed to expand in neuroblastoma (NB) cells in vitro (examined 7 dpi). Merged fluorescence image superimposed with phase-contrast image were demonstrated. Consequently, while rOkaD7 entered human DRG xenografts in culture, it failed to replicate in human DRG xenografts, cultured human DRG or in neuroblastoma cells in vitro. This evidence indicated that the ORF7 is a novel VZV skin- and neurotropic factor.

### Example 3

Antibodies raised against VZV ORF7 protein appear to bind to a 29-kDa protein localized in the trans-golgi network of rOka-infected cells and incorporated into VZV virions. To determine the role of ORF7 in neuronal cell infection in one exemplary method, viral entry, DNA replication, gene expression, capsid assembly/cytoplasmic envelopment, and transcellular transmission were compared in cells infected with rOka, rOkaD7 (D7) and D7 rescue virus (R7) to assess the role of ORF7.

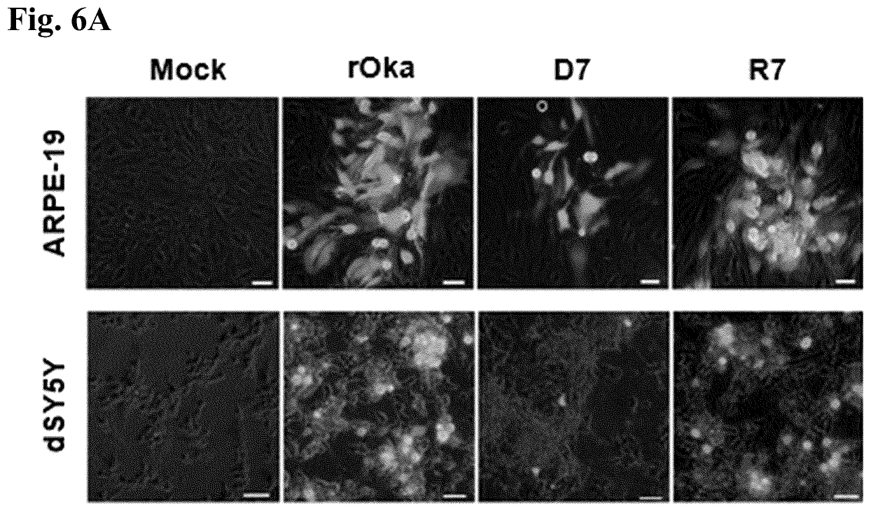

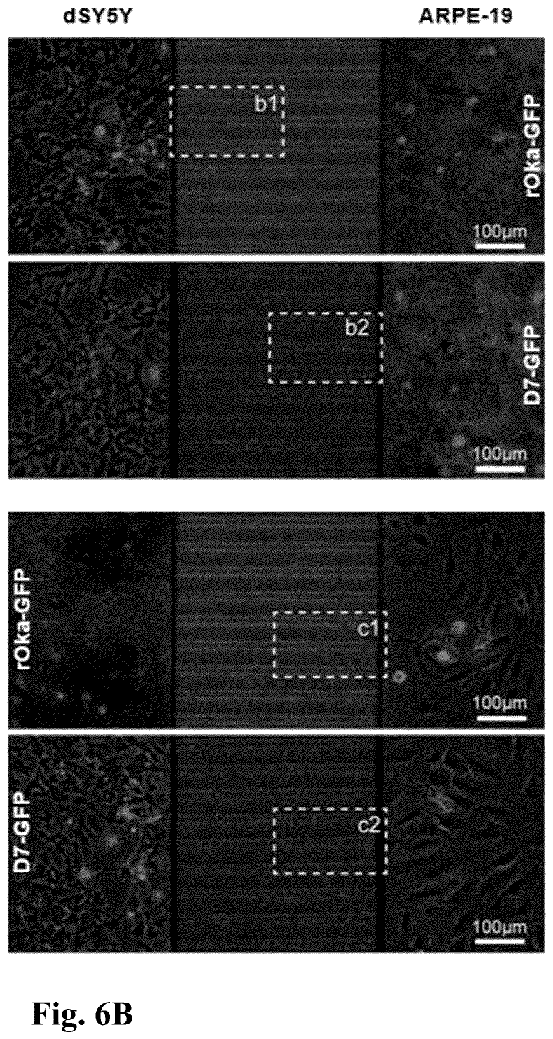

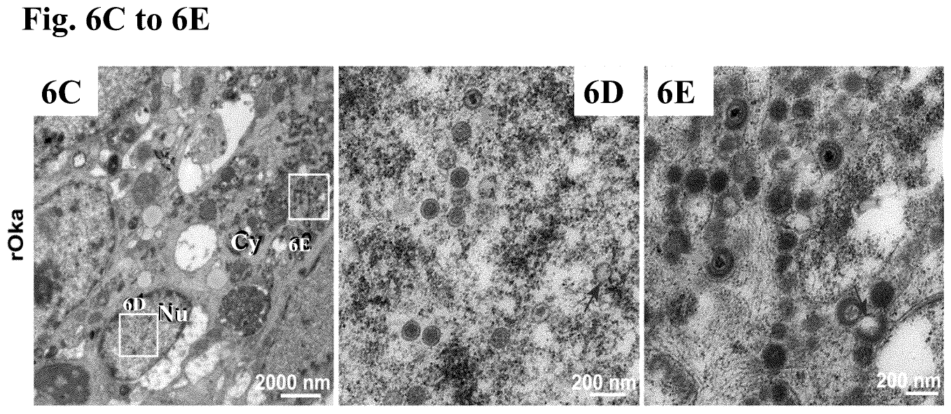

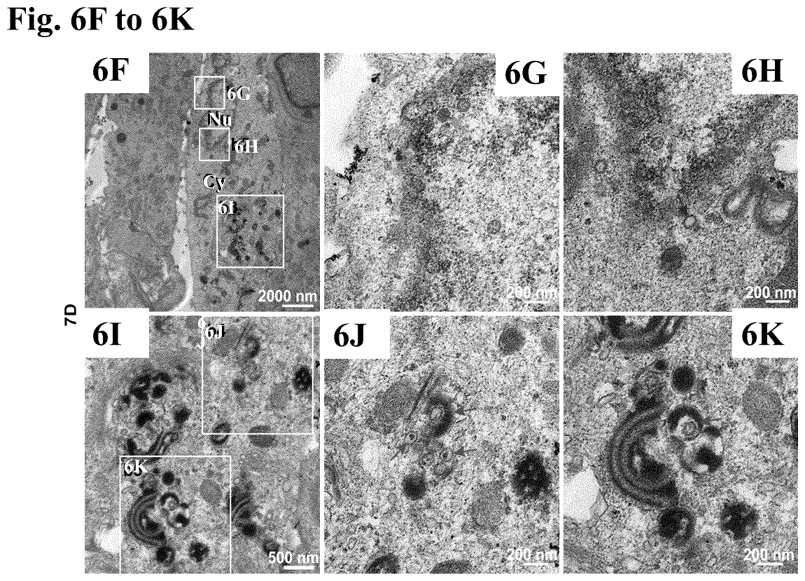

As discovered herein, D7 virus was found to be expressed its genes in dSY5Y but was not transmitted to adjacent neurons as illustrated in exemplary experiments using fluorescent indicators (FIG. 6A). In another experiment, microfluidic chambers were used to assess the ability of D7 virus compared to a wild-type virus to spread axonally. Both rOka-GFP and D7-GFP viruses were transmitted from ARPE-19 (Adult Retinal Pigment Epithelial cell line-19) to dSY5Y (differentiated SH-SY5Y human neuroblastoma cells), but D7-GFP failed to spread axonally in a microfluidic chamber mimicking in vivo transmission (FIG. 6B, upper two panels). In dSY5Y infection, only rOka crossed the chamber and infected ARPE-19 cells (FIG. 6B, bottom two panels) illustrating in vivo transmission. Using electron microscopy, the D7 defect was observed to be during viral cytoplasmic envelopment, because VZV particles were observed in the nucleus (FIGS. 6C and 6D capsids with genome, green arrow; capsids without genome, blue arrow) and in the cytoplasm (FIGS. 6C and 6E, intact virion, green arrow; hollow particle, blue arrow) of rOka-infected dSY5Y cells, whereas nucleocapsids were observed in the nucleus (FIGS. 6F to 6H capsids with genome, green arrow; capsids without genome, blue arrow) of D7-infected dSY5Y cells, but envelope-defective particles (red arrows) were captured in the cytoplasm (FIGS. 6I to 6K). These exemplary experiments provide support that envelopment deficiency caused by ORF7 deletion leads to limited dissemination of VZV among neuronal cells indicating that this region is responsible for this distribution and without it the virus has significantly reduce to completely eliminated distribution to neuronal cells.

### Example 4

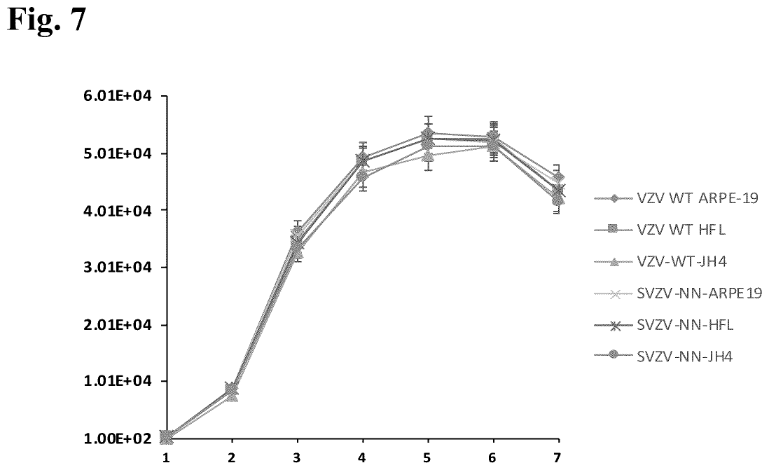

In another exemplary method, replication of VZV and SVZV-NN (prepared as described in Example 1) was compared in human fibroblasts (HFL), human retinal pigment epithelial (ARPE-19) and guinea pig fibroblast (JH4) cells. Briefly, ARPE-19, HFL and JH4 cells were seeded into six well plates. The cells were then infected with VZV and SVZV-NN at MOI of 0.01, and growth curve analysis was performed. The growth of VZV and SVZV-NN were comparable in all three cell lines, indicating that SVZV-NN does not have noticeable growth defect (FIG. 7).

### Example 5

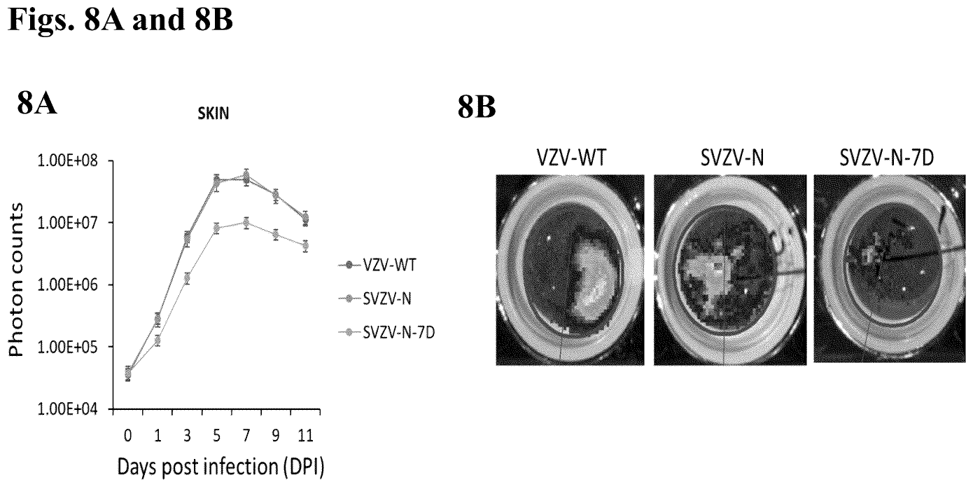

In another exemplary method, human fetal skin samples (20 weeks gestational age) were cultured in a skin organ culture medium to analyze skin infectivity of various viruses in comparison to control viruses. Briefly, the skin tissues were infected with 5×10³ plaque-forming units (PFU) per tissues. After infection, the tissues were placed in 12 well plate with 500 μm mesh Net Well inserts. The tissues were rested above 1 ml of skin organ culture medium. The infected skin samples were incubated with 150 μg/ml of d-luciferin for about 10 minutes followed by imaging (for example using an IVIS imaging system) to determine the photon counts. The skin tissues samples were placed back in fresh culture medium. FIG. 8A illustrates an exemplary growth curve tracking viral replication in the skin samples from 0 to 11 dpi, visualized in representative fluorescent images demonstrating viral infectivity of the skin cells as plated (See FIG. 8B) SVZV-N-7D growth was attenuated but not completely eliminated in the skin samples.

### Example 6

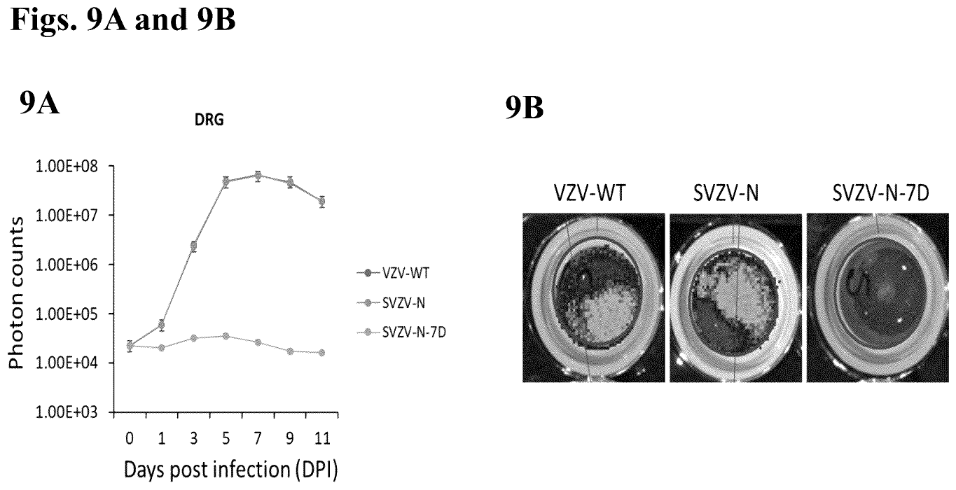

In another exemplary method, human fetal DRGs were isolated from 20-weeks-old spinal cord samples. DRGs were disinfected with alternative application of 70% ethanol and isopropyl alcohol. Then, infection was carried out using 100 PFU/well of cell free virus. Luciferase activity was measured every 48 hours to assess viral infectivity of the DRG cells. FIG. 9A illustrates an exemplary growth curve tracking viral replication in these cells for VZV-WT, SVZV-N and SVZV-N-7D viruses. FIG. 9B illustrates representative merged images of the different viruses replicating in DRG samples. As demonstrated, SVZV-N-7D had significantly reduced infectivity in these cells relative to viruses having intact ORF7.

### Example 7

In another exemplary method, SVZV chimeric viruses constructed in Example 1 were used to study varicella pathogenesis in NHPs. In another method, African green monkeys (AGM) were used to study viral infectivity. In this example, the AGMs were inoculated intravenously (i.v.) with chimeric virus SVV-LE-NN, SVV-RE-NN and control rOka VZV. As shown in Table 3, only AGM inoculated with SVV-LE-NN (monkeys 1 and 2) developed clinical signs of varicella and rash at 14 days post-inoculation (dpi) (Table 4).

TABLE 4

| | | Days post infection (dpi) | | | | | |
|---|---|---|---|---|---|---|---|
| | | 14^{a} | | | | 100^{a} | |
| Monkey | | skin | blood | lung | lung | lung | ganglia |
| number | Virus | rash | (PCR) | (PCR) | (IHC) | (PCR) | (PCR) |
| 1 | SVZV-NN | yes | + | + | + | nd | nd |
| 2 | SVZV-NN | yes | + | nd^{b} | nd | − | + |
| 3 | SVZV-RE-NN | no | − | nd | nd | − | − |
| 4 | VZV | no | − | nd | nd | − | + |
| 5 | SVV^{c} | yes | + | + | + | − | + |

^{a}based on Southern blot analysis (sensitivity: 10-100 copies/μg of DNA)
^{b}not done;
^{c}previously published data

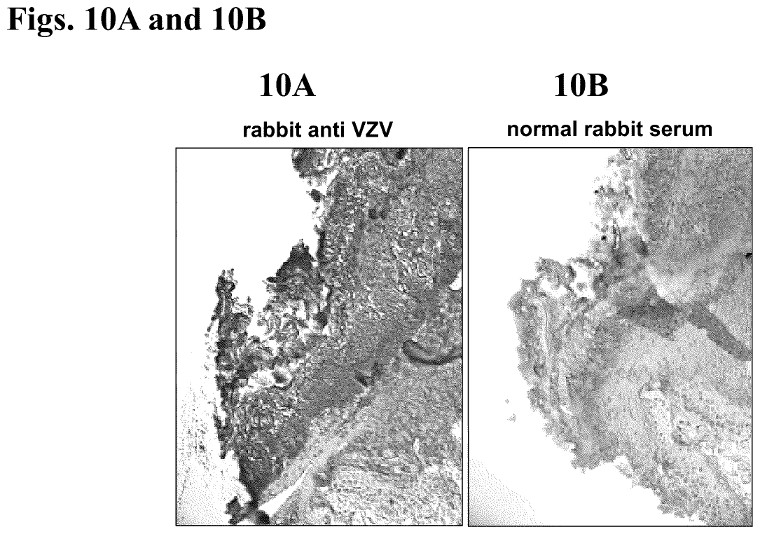

These data demonstrated the ability to use this construct as a viral model in a non-human primate for additional studies. In these examples, Monkey 1 was euthanized at the time of varicella rash (14 dpi), with the remaining monkeys euthanized at 100 dpi. VZV DNA was detected by PCR in PBMC from monkeys 1 and 2 and in lung from monkey 1 at the time of acute infection. VZV antigens were detected using IHC in sections of skin during varicella in monkey 1 (Table 4, FIGS. 10A and 10B).

VZV DNA was detected in ganglia of monkey 2 (inoculated with SVV-LE-NN) and monkey 4 (inoculated with rOka VZV), but not in ganglia of monkey 3 (inoculated with SVV-RE-NN) at 100 dpi. Lung samples from monkeys 2, 3 and 4 were negative for VZV DNA at 100 dpi. These results confirm that SVZV-NN infects AGMs, produces skin rash, and enters DRG. It was also demonstrated that SVV-LE-NN virus becomes latent in monkeys, based on the detection of virus DNA in ganglia but not in lung at 100 dpi (Table 4). Wild-type Oka VZV inoculated i.v. into monkeys did not produce skin rash, but VZV DNA was found in ganglia at 100 dpi. This detection of VZV DNA in ganglia reflects easier access to ganglia by i.v. inoculation, in the absence of clinical disease. These findings suggested that NHP infected with SVZV chimeras are a useful animal model to study VZV (as well as other human viruses) pathogenesis and latency.

### Example 8

In another exemplary method, VZV does not produce disease in NHP, however, SVV infection of NHP recapitulates most aspects of human infection. VZV is an exclusively human, neurotropic herpesvirus that infects and remodels the surface architecture of T cells for its transport to skin. Wild type (wt) SVV infection in NHP produces viremia with infection of multiple immune cells, including T-cells, infection of which appears to be critical for hematogenous viral dissemination. The data in Table 4 shows that SVZV-NN infected and caused rash in NHP and became latent in ganglia. Based on these data, multiple SVZVs are used to test the hypothesis that the insertion of SVV-specific sequences into the VZV genome enables dissemination of virus by infected T-cells in NHP. Specifically, the SVZV-NN and SVZV-N construct described in the examples above are used to characterize T-cell infection, extent of dissemination and disease as compared to wtSVV- and rOkaVZV-infected controls. In addition, SVZV-NN-D7 and SVZV-N-D7 are tested in monkeys to determine the role of ORF7 in T-cell infection and production of varicella.

Inoculation of rhesus macaques with SVZV. Wild-type VZV (rOkaVZV), SVV (wtSVV) and SVZV-LE-NN, SVZV-LE-NN-D7, SVZV-LE-N, and SVZV-LE-N-D7 (constructed and characterized in Example 1) are propagated in rhesus fibroblasts. A total of 40 rhesus macaques (equal numbers of male and female) are inoculated with the 4 SVZVs (8 monkeys/virus) and with rOkaVZV (4 monkeys) and wtSVV (4 monkeys). After anesthesia for all procedures, animals are intratracheally inoculated with 1×10⁶ PFU of each virus. The underside of SVV-seronegative rhesus macaques are shaved and monitored for physical and clinical parameters including: weight, temperature, physical condition, lymph node (LN)/spleen palpation, and rash. Blood and saliva are collected and skin is examined for rash at 0, 4, 7, 9, 11 14, 17, 21, 28, 42, 56 and 90 dpi. Complete blood counts (CBC) and serum chemistry analyses are performed monthly. Liver-specific aminotransferase assays of blood at 0, 10, and 28 dpi is used to confirm hepatic infection. BAL samples are obtained at 0, 4, 7, 9, 14, and 28 dpi. LN biopsies are obtained at 0, 7 and 11 dpi.

Plasma is analyzed for SVV- and/or VZV-specific antibodies by serum neutralization assay on days 28 and 56. Widespread varicella rash is typically observed at 9-14 dpi and expected in monkeys inoculated with wtSVV, SVZV-LE-NN and SVZV-LE-N. Skin rash is scraped with a sterile scalpel for dispersal in sterile saline. Total DNA extracted from saliva, blood, BAL and skin scrapings is analyzed for SVV- and VZV-specific DNA sequences by real-time qPCR. Punch biopsies of the area of rash are also collected for histopathologic, IHC and in situ (ISH) analyses.

Analysis of skin scrapings and punch biopsies. Skin scrapings are divided into two portions for: 1) DNA extraction and PCR analysis (see below); and 2) co-cultivation with indicator cells to recover replicating SVZV for comparison with the inoculum. Total DNA is extracted from skin scrapings and analyzed by qPCR. Punch-biopsies are paraformaldehyde (PFA)-fixed, paraffin-embedded and stained with hematoxylin and eosin for the presence of intranuclear inclusion bodies. IHC is performed as previously described using rabbit polyclonal anti-VZV antibodies. Detection of late VZV antigens confirms VZV replication in rash.

Assessment of virus infection in T-cells. Viremia is assessed in PBMC, LN and BAL by co-cultivation with Vero cells. Flow cytometry and real-time PCR of DNA from PBMC, BAL and LN confirms the expected infection of alveolar macrophages, dendritic cells and transfer of virus to T-cells in LNs. T and B lymphocytes, monocytes and dendritic cells are separated with combinations of fluorochrome-conjugated monoclonal antibodies as demonstrated below.

Unfixed and stained cells are sorted four-ways using a FACS-Fusion cell sorter instrument with at least 1-1.5×10⁶ cells sorted for each subset. Identification of the virus-infected cell population allows re-sorting with more detailed antibody sets to test for the infected subpopulation. T-cells are the expected major cell population infected and are further sorted with antibodies to CD4, CD8, CD28 and CD95 that identify differential infection in either CD4 or CD8 naïve, effector and central memory populations. A minimum of 1×10⁶ viable cells in qPCR is used to determine the extent of virus infection.

Analysis of innate, humoral, and cellular immune responses. Virus-specific immune responses in blood, BAL, and LN in infected NHP are analyzed as follows.
(i) Innate cytokine responses: Innate responses in sera of SVZV, VZV or SVV-infected rhesus macaques are compared in a head-to-head manner with a Multiplex Luminex® bead assay based on xMAP® technology. Response measured are specific for NHP cytokines that determine levels of 29 cytokines, chemokines, and growth factors. Assays are quantified for example, on the Bio-Plex® 200 Suspension Array System and compared with expected cytokine results.
(ii) Humoral immune responses: Antibody responses to SVV and VZV are assessed by virus-specific functional plaque-reduction assays (PRNT) and ELISAs for all infected NHP.
(iii) Cell-mediated immune responses: Peripheral blood, BAL and LN collected from all NHP at various times are processed and examined by 13-color flow cytometry to determine the phenotype and dynamics of cell populations during infection, using a BD FACS Fortessa flow cytometer capable of 15-parameter analysis, and analyzed with FlowJo software. Also used is BD FACS Symphony, which is capable of 28-color analysis. A single panel is developed that includes all markers of interest along with other markers enabling deep analysis with fewer cells. Lymphocyte subsets, and associated memory subpopulations are quantified and patterns compared with wtSVV patterns according to Table 5.

TABLE 5

| | | | PE- | | PE- | Pac | | | | | | | Cy7- |
|---|---|---|---|---|---|---|---|---|---|---|---|---|---|
| Panel | FITC | PE | TxRed | Cy5.5PerCP | Cy7 | Blu | BV510 | BV605 | BV650 | BV11 | APC | A/700 | APC |
| A | CD4 | CD16 | HLA-DR | CD123 | CD3 | CD14 | Live/Dead | CD45 | CD8 | CD95 | CD1c | CD11c | CD20 |
| B | CXCR5 | CCR5 | CD8 | CD28 | CCR7 | CD34 | Live/Dead | CD45 | CD3 | CD95 | CD103 | CCR2 | CD4 |
| C | KI67 | FoxP3 | CD8 | HLA-DR | CD25 | CD34 | Live/Dead | CD45 | CD3 | CD62L | CD38 | CD69 | CD4 |

| AIM | | | PE- | | PE- | | | |
|---|---|---|---|---|---|---|---|---|
| Panel | FITC | PE | CF594 | Cy5.5PerCP | Cy7 | BV421 | BV510 | BV605 |
| D | CD25 | CXCR5 | CD28 | CD4 | 4-1BB | OX40 | Live/Dead | CD3 |

| AIM | | | | | Cy7- | | |
|---|---|---|---|---|---|---|---|
| Panel | BV650 | BV711 | APC | BUV396 | APC | BUV496 | BUV737 |
| D | CD14 | CD95 | PD1 | CD45 | CD20 | CD16 | CD8 |

Panel A: Immune cell phenotyping
Panel B T cell memory subsets identification
Panel C: T cell memory subset activation and proliferation
Panel D: AIM assay

Absolute counts of all T- and B-cell subsets are calculated by incorporating CBC count. The expected robust B- and CD4 T-cell and lymphoid memory cell responses to SVZVs, SVV and VZV are characterized. B- and T-cells are tracked with the Ki67 proliferation marker, CD4+ and CD8+ T cells and their associated naive, central memory and effector memory populations: CD28+CD95−, CD28+CD95+, and CD28−CD95+in PBMC, BAL and LN. Virus-specific response are analyzed by stimulation with cell lysates from either SVV or VZV infected cells, vaccinia infected cells (negative control) using 1 μg/10⁶ PBMC and CD3 (positive control, 0.1 μg/well), stained with appropriate memory surface antibodies and CD107a to assess cytotoxic potential, fixed, permeabilized and then stained for cytokines, IFNγ and TNFβ.

Statistical Analyses: SVZV-NN and SVZV-N produce high viremia indicative of acute infection in all 16 macaques. Differences in biological samples of blood, BAL and saliva at 0 -90 dpi is assessed using descriptive statistics (mean ±SD). A randomized block analysis of variance (ANOVA) model is used to compare the 6 different experimental groups at each time point. Pairwise comparisons are used to identify which groups differ from one another and specific controls. The false discovery rate is used to adjust for multiple comparisons. Repeated measures ANOVA are used to compare changes over time in the treatment groups. Biological samples from NHPs infected with SVZV-NN and SVZV-N are compared to those from the wtSVV wild-type; SVZV-NN-d7 and SVZV-N-d7 are compared to the wtVZV. A permutation F-test is used for the small sample size to avoid assuming asymptotic normality. If the observation sizes exceed the sample size (n>p where matrices are not full rank), and many markers are combined in the same analysis, regularization regression methods are used because they can handle high-dimensional and highly correlated data. These methods are used to identify the most important differentiating indicators of infection based on treatment group membership. ANOVA model for statistical analysis, with power was calculated based on the F-statistic using an α=0.05. Comparing SVZV-NN to SVZV-NN-D7 or SVZV-N to SVZV-N-D7 with 8 animals per group provides a power of 0.96 to detect significant differences. The effect size estimate is based on the mutant viruses producing 10% of the viral DNA and RNA copy numbers of that of the wild-type virus, with a standard deviation of 5%. Significant differences in viral DNA copy number shows corresponding differences of similar magnitude in measures of cytokines and antibodies after establishment of latency.

Experimental Outcomes. SVZV-LE-NN and SVZV-LE-N DNA is detected in PBMCs 4 dpi, peaking at 7-10 dpi and returning to baseline by 14 dpi. SVZV DNA is present in macrophages, dendritic cells, NK cells, but mostly in T-cells, as are pro-inflammatory cytokine responses. VZV-specific antibodies are in sera ˜14 dpi, with levels remaining high thereafter. Infection of central and effector memory T-cells and lower levels, if any, of viremia is in NHP inoculated with rOkaVZV. Because ORF7 deletion mutants of VZV are deficient only in skin and neuronal spread, the DNA specific for SVZV-NN-D7 and SVZV-N-D7 are found in blood. At 90 dpi, higher levels (10-50 copies/μg) of wtSVV, SVZV-NN and SVZV-N DNA are present, and much lower levels (1-5 copies/μg) of DNA are found for SVZV-NN-D7, SVZV-N-D7 DNA, and limited amounts, if any, of rOkaVZV DNA in ganglia. No virus DNA seen in lung or liver in all virus infections confirms latency. The results herein provide the groundwork for construction and testing of additional SVZV mutants containing subfragments of the 3-kb segment located at the left end of the genome.

### Example 9

In another exemplary method, SVZV chimeras generated herein provide the ability to determine if ORF7 mutant enters and establishes latency in ganglia but does not infect other neurons, and to identify other neurotropic factors. The VZV ORF7 deletion mutant does not infect human skin and neurons ex vivo and in vivo. The chimeric virus SVZV-NN produces skin rash in monkeys (Table 4). SVZV mutants NN-D7 and N-D7, which contain truncations in ORF7 coding sequences, are used to infect rhesus macaques similar to the methods described in Example 8. Analysis of ganglia from these monkeys at 90 dpi in this example determines that VZV ORF7 is essential for latency in ganglia.

Analysis of ganglionic and non-ganglionic tissues. Following the methods described in Example 9, 16 of the 40 rhesus macaques are inoculated with SVV ORF7 mutants. All NHPs, including controls, are inoculated, monitored for infection and euthanized at 90 dpi. Blood, skin, lungs, liver, spleen, adrenal glands, and ganglia are collected and analyzed as in Example 9.

PBMC and ganglia are analyzed for virus DNA and RNA as described in the examples herein. Paraffin-embedded tissues are examined by ISH for VLAT and DapB negative control probe using the ACD (Advanced Cell Diagnostics) ISH system and by IHC using VZVORF63 and glycoprotein E antibodies.

Statistical analysis. Similar to Example 9, a randomized block permutation F test makes the comparisons in the 6 treatment groups. SVZV-NN-D7 and SVZV-N-D7 show evidence of latency with a very low virus DNA copy number. A permutation-based ANOVA is used to assess differences in viral loads in the tissues collected, and for virus DNA and RNA in PBMC and ganglia and use regularization methods for assessing the importance of any subset of significant indications of latency identified as described.

### Example 10

It is noted that preclinical trials of HIV vaccines using multiple vaccine platforms and delivery systems that have been tested in nonhuman primates are not able to use HIV due to the inability of HIV to replicate in a nonhuman primate. Instead, as disclosed herein, multiple vaccines studies use the simian counterpart virus, SIV (simian immunodeficiency virus) as a surrogate virus for improved testing.

In these methods, SIV preclinical trials are performed using an SIV vaccine platform which includes a recombinant SVV backbone with inserted fragments of for example, SIV genes (rSVV-SIV) to induce immunity to SIV in the non-human primate. This recombinant viral construct that directly utilizes the HIV genome with the inserted nucleic acid sequence fragment of simian varicella virus (SVV) is used to infect these non-human primates for direct testing of antiviral drugs and vaccines on the actual HIV genes/genome. Methods of making the viral construct and infecting non-human primates are similar to those described in Examples 1-9 herein.

### Example 11

In another exemplary method, gene therapy applications are disclosed. Development of a gene therapy delivery system using a viral vector containing a VZV ORF7 (with or without stop codons) (or an SVZV chimera containing ORF7) results in constructs that target the skin and/or the dorsal root ganglion of a human subject and delivers appropriate genes or other agents that treat or correct targeted skin or dorsal root ganglion genetic defects or localized health conditions. It is noted that these gene therapy delivery applications herein can be first tested in non-human primates. In one exemplary method, an example defect in the dorsal root ganglia corrected using these gene delivery systems herein is delivering a replacement Frataxin (FXN) gene which is defective in the conditioned referred to as Friedreich's ataxia (FRDA). FRDA is an autosomal recessive neurodegenerative and cardiac disorder with an estimated incidence of 1:29,000 to 50,000. FRDA produces dorsal root ganglia and peripheral nerve degeneration causing such conditions as: progressive limb and gait ataxia, loss of postural balance, oculomotor dysfunction and cardiomyopathy. There is currently no effective treatment but would be a likely candidate for gene therapy. Gene therapy systems herein have tropism for neurological tissues and lead to targeted tissue expression of FRDA to treat this condition. Another example of gene delivery with the viral chimera constricts herein include gene therapy systems that target gene-associated with pain reduction in postherepetic neuralgia.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A chimeric viral construct, wherein the chimeric viral construct comprises a nucleic acid sequence from an exclusively human pathogenic virus and a nucleic acid sequence fragment of simian varicella virus (SVV), wherein the nucleic acid sequence fragment of SVV comprises 85% or more sequence identity to SEQ ID NO: 1.

2. The chimeric viral construct according to claim 1, wherein the chimeric viral construct comprising the nucleic acid sequence from an exclusively human pathogenic virus comprising a nucleic acid sequence of an adenovirus, an anellovirius, an astrovirus, a calicivirius, a coronavirus, a flavivirus, a herpesvirus, an orthomyxovirus, a papillomavirus, a paramyxovirus, a parvovirus, a picornavirus, a polyomavirus, a poxvirus, a pneumovirus, a retrovirus, or any combination thereof

3. The chimeric viral construct according to claim 1, wherein the chimeric viral construct comprising the nucleic acid sequence from an exclusively human pathogenic virus comprises a nucleic acid sequence from human herpes simplex virus type 1 (HSV-1), herpes simplex virus type 2 (HSV-2), Epstein-Barr-virus (EBV), cytomegalovirus (CMV), human herpes virus type 6 (HHV-6), human herpes virus type 7 (HHV-7), human herpes virus type-8 (HHV-8), varicella zoster virus (VZV), or any combination thereof.

4. The chimeric viral construct according to claim 1, wherein the chimeric viral construct comprising the nucleic acid sequence from an exclusively human pathogenic virus comprises a nucleic acid sequence derived from VZV.

5. A non-human primate model comprising, the non-human primate infected with a chimeric viral construct comprised of a nucleic acid sequence from an exclusively human pathogenic virus and a nucleic acid sequence fragment from simian varicella virus (SVV) according to claim 1.

6. The non-human primate model according to claim 5, wherein the chimeric viral construct comprising the nucleic acid sequence from the exclusively human pathogenic virus comprises a nucleic acid sequence from an adenovirus, an anellovirius, an astrovirus, a calicivirus, a coronavirus, a flavivirus, a herpesvirus, an orthomyxovirus, a papillomavirus, a paramyxovirus, a parvovirus, a picornavirus, a polyomavirus, a poxvirus, a pneumovirus, a retrovirus, or any combination thereof.

7. The non-human primate model according to claim 5, wherein the chimeric viral construct comprising the nucleic acid sequence from an exclusively human pathogenic virus comprises a nucleic acid sequence from human herpes simplex virus type 1 (HSV-1), herpes simplex virus type 2 (HSV-2), Epstein-Barr-virus (EBV), cytomegalovirus (CMV), human herpes virus type 6 (HHV-6), human herpes virus type 7 (HHV-7), human herpes virus type-8 (HHV-8), varicella zoster virus (VZV), or any combination thereof.

8. The non-human primate model according to claim 5, wherein the chimeric viral construct comprising the nucleic acid sequence from an exclusively human pathogenic virus comprises the nucleic acid sequence from VZV.

9. (canceled)

10. The non-human primate model according to claim 5, wherein the non-human primate is a rhesus macaque, a cynomolgus macaque, an African green monkey, a baboon, a sooty mangabey, a common marmoset, a capuchin, an owl monkey, a patas money, a pigtail macaque, a sabaeus monkey, a squirrel monkey, or a tamarin.

11. The non-human primate model according to claim 5, wherein the chimeric viral construct comprises a nucleic acid sequence of a human VZV.

12. The non-human primate model according to claim 11, wherein the nucleic acid sequence of a human VZV comprises at least 3 stop codons in open reading frame 7 (ORF7) of the VZV nucleic acid sequence.

13. A method of making the chimeric viral construct according to claim 1, the method comprising using a recombineering system to introduce the nucleic acid sequence fragment of SVV into one or more locations within the nucleic acid sequence from an exclusively human pathogenic virus.

14. The method of making the chimeric viral construct according to claim 13, wherein the nucleic acid sequence fragment of SVV is introduced into at least one of an N-terminus of the nucleic acid sequence from an exclusively human pathogenic virus, a C-terminus of the nucleic acid sequence from an exclusively human pathogenic virus, a region between the N-terminus and the C-terminus of the nucleic acid sequence from an exclusively human pathogenic virus.

15. The method of making the chimeric viral construct according to claim 13, wherein the nucleic acid sequence fragment of SVV is introduced to at least one of an N-terminus of the nucleic acid sequence of a human VZV, a C-terminus of the nucleic acid sequence of a human VZV, a short sequence (Us) of the nucleic acid sequence of a human VZV, a nucleic acid segment between open reading frame 65 (ORF65) and open reading frame 66 (ORF66) of a nucleic acid sequence of a human VZV, or any combination thereof.

16. A method of making a non-human primate model, the method comprising infecting a non-human primate with one or more of the chimeric viral constructs according to claim 1.

17. (canceled)

18. An in vivo screening system for antiviral agents, the in vivo screening system comprising:
a non-human primate model infected with a chimeric viral construct according to claim 5, and,
at least one antiviral agent for use to treat or prevent infection in the non-human primate model of the exclusively human pathogenic virus for in vivo screening of the at least one antiviral agent in the non-human primate model.

19. The in vivo screening system according to claim 18, further comprising at least one assay, wherein the assay comprises reagents for measuring at least one of positive and negative output from the in vivo screening system for assessing efficacy of the antiviral agent against the exclusively human pathogenic virus in the non-human primate model.

20-21. (canceled)

22. A gene therapy delivery system, the system comprising a vector, the vector comprising,
a nucleic acid sequence of open reading frame 7 (ORF7) of human VZV, the ORF7 comprising 85% or more sequence identity to SEQ ID NO: 8, and
a nucleic acid sequence encoding at least one therapeutic or replacement gene,
wherein the at least one therapeutic or replacement gene is expressed in a neuronal cell, a dermal cell, or a combination thereof.

23. The gene therapy delivery system according to claim 22, wherein the vector further comprises a viral vector, the viral vector comprising a retrovirus, an adenovirus, an adeno-associated virus, a lentivirus, a pox virus, an alphavirus, a herpes virus, or any combination thereof.

24-25. (canceled)

26. A method of treating a disease or a condition in a subject, the method comprising administering a gene therapy delivery system according to claim 22 to the subject.

27. (canceled)

28. A kit comprising at least one of the chimeric viral constructs according to claim 1 and at least one container.

29. (canceled)
